(19) United States
(12) Patent Application Publication
(10) Pub. No.: US 2026/0283990 A1
(43) Pub. Date: Sep. 24, 2026

(54) COMPOUNDS FOR TREATING RETT SYNDROME AND OTHER DISORDERS

(71) Applicant: UNRAVEL BIOSCIENCES, INC., BOSTON, MA (US)

(72) Inventors: RICHARD NOVAK, BOSTON, MA (US); FREDERIC VIGNEAULT, BOSTON, MA (US); ERICA GARDNER, BOSTON, MA (US); SEVGI REAGH, BOSTON, MA (US); RAHUL NIHALANI, BOSTON, MA (US)

(21) Appl. No.: 19/473,875

(22) PCT Filed: Apr. 12, 2024
(86) PCT No.: PCT/US2024/024186

§ 371 Date: Oct. 9, 2025

Related U.S. Application Data
(60) Provisional application No. 63/458,704, filed on Apr. 12, 2023.

(51) Int. Cl. A61K 31/196; A61K 31/167; A61K 31/421; A61K 31/4245; A61K 31/426; A61K 31/437; A61K 31/5025; A61P 25/28

(52) U.S. Cl. CPC A61K 31/196; A61K 31/167; A61K 31/421; A61K 31/4245; A61K 31/426; A61K 31/437; A61K 31/5025; A61P 25/28

(57) ABSTRACT

The subject invention pertains to compositions comprising compounds engaging short fatty acid Co-enzyme A (CoA) metabolism, including short fatty acid Co-A synthetase 3 (ACSS3), and methods of treating Rett Syndrome (RTT) and other neurodevelopmental and metabolic disorders comprising administering the subject compositions to a subject in need thereof. In preferred embodiments, the compound may comprise succinanilic acid or another compound as disclosed herein. The disclosed compounds can engage a majority of the identified targets and mechanisms, including, for example, altering the expression of genes encoding RUNX3, MeCP2, or proteins in the KEAP1-NFE2L2 pathway without inhibiting HDACs.

### CROSS-REFERENCE TO A RELATED APPLICATION

This application claims the benefit of U.S. Provisional Application Ser. No. 63/458,704, filed Apr. 12, 2023, the disclosure of which is hereby incorporated by reference in its entirety, including all figures, tables and amino acid or nucleic acid sequences.

### BACKGROUND OF THE INVENTION

Rett Syndrome (RTT) is a rare and severe genetic neurodevelopmental disorder (NDD) with no approved treatment and is a leading cause of cognitive disabilities in females. The disease was first described 55 years ago by Andreas Rett (Rett, 1966). 23 years ago, the loss of function of the gene encoding methyl-CpG binding protein (MeCP2), a ubiquitous transcriptional regulator located on the X chromosome, was identified as the genetic cause (Amir et al., 1999). RTT affects 1 in 10,000 females (Fehr et al., 2011; Anderson et al., 2014) and is a considered a disorder with high unmet needs, along with other neurological disorders due to limited existing treatments, and the difficulty in treating all the sites affected by their gene deficiency such as the central nervous system (CNS), peripheral nervous systems (PNS) and adjacent systems, such as digestive, respiratory, muscular, skeletal, and immune. The first symptoms present at 6 to 18 months of age and are characterized by a regression in developmental milestones including language, motor coordination and mental retardation (Frullanti et al., 2019). These progress in severity over time and depending on the mutations, lead to partial or complete loss of speech, hand movements, and gait, and often include seizures, scoliosis, growth delay, breathing and gastrointestinal problems and more. While patients can live into their 60s (Kerr and Prescott, 2005; Tarquinio et al., 2015; Henriksen et al., 2020), the burden of care is significant, requiring 24-hour care (Fu, Armstrong, Marsh, Lieberman, Motil, Witt, Standridge, Nues, et al., 2020) with medical costs up to $165,000/year, depending on the severity, at an average of $41,000/year (inflation and dollar adjusted) (Hendrie et al., 2011). Although this is an orphan disease, it results in an estimated $680M in additional healthcare costs in the United States. While our understanding of the disease has greatly improved, even after decades of research, there are still no approved treatments for RTT.

Clinical criteria and research have primarily focused on the neuromotor, social, and cognitive impairments, with essential criteria listing regression, loss of speech, seizures, microcephaly, and autistic features (Hendrie et al., 2011; Buchanan et al., 2019; Fu, Armstrong, Marsh, Lieberman, Motil, Witt, Standridge, Nues, et al., 2020). However, RTT patients present with broad clinical symptoms affecting multiple systems, including respiratory dysfunction, gastrointestinal issues, swallowing difficulty, sleep disturbances, bone density, nociception derangement, low-level systemic inflammation, and a shortened lifespan (Ellaway et al., 2001; Hagberg, 2002; Katz et al., 2012; De Felice et al., 2014; Tsuchiya et al., 2015; Pecorelli et al., 2016, 2020; Kyle, Vashi and Justice, 2018; Vashi and Justice, 2019; Fu, Armstrong, Marsh, Lieberman, Motil, Witt, Standridge, Lane, et al., 2020; Wang et al., 2021; Wong et al., 2021; Zhang, Li and Spruyt, 2021). Currently, the diagnosis and characterization of a RTT patient is determined by a phenotypic scoring method consisting of a series of clinical features to account for the heterogeneity of the patient population (Kerr et al., 2001; Colvin et al., 2003; Fehr et al., 2011). Furthermore, none of the drugs currently in clinical trials have been developed to tackle cognitive function due to a lack of cognitive screens in early drug development.

Heterozygous, de novo mutations in the MECP2 gene, located at Xq28, cause RTT (Amir et al., 1999). Girls affected by RTT display a random pattern of X-chromosome inactivation (XCI) and are mosaic for MeCP2 expression in all tissues, which contributes to the diverse presentation and severity of RTT phenotypes (Nielsen et al., 2001; Braunschweig et al., 2004; Zhang et al., 2012; Wither et al., 2013; Rietveld et al., 2015; Renthal et al., 2018). MeCP2 is a 486-amino acid nuclear protein, that binds to methylated CpG dinucleotides to influence the expression of thousands of genes, acting as both transcriptional repressor and activator for ~25% of our genes (Chahrour et al., 2008; Krishnaraj et al., 2019). The protein contains a methyl binding domain (MBD) which binds to DNA, and a transcriptional repressor domain (TRD), with the C-terminal portion containing the NCoR/SMRT interaction domain (NID) (Lyst et al., 2013). Over 900 distinct loss-of-function mutations in MeCP2 have been reported in patients with RTT (Anitha et al., 2022), most occurring within exons 3 and 4 of the gene, a segment less than 2.5 Kbp in size, and have been shown to be associated with the clinical presentation and severity (Amir et al., 1999; Cuddapah et al., 2014; Tsuchiya et al., 2015; Shovlin and Tropea, 2018; Ribeiro and MacDonald, 2020; Ehrhart et al., 2021).

Furthermore, there is a disconnect between pre-clinical and clinical evaluation of therapeutic interventions contributing to poor clinical success. The development of well-characterized murine models has greatly improved our understanding of RTT and enabled drug studies in animal models with symptoms relevant to human patients, including motor and cognitive function disruption (Guy et al., 2001; M. D. Shahbazian et al., 2002; M. Shahbazian et al., 2002; Wu et al., 2016). These studies have been limited by the predominance of the C57BL/6J mouse strain and the difficulty in accessing non-human primate models. This limits the throughput needed for comprehensive new drug R&D, while demonstrating pre-clinical efficacy in RTT murine models have not translated to the clinic (Katz et al., 2016; Vashi and Justice, 2019; Borloz, Villard and Roux, 2021; Ivy and Standridge, 2021). Endpoints have focused on central nervous system (CNS) symptoms, while non-CNS endpoints seen in models and patients have been slow to be adopted in clinical trials. Additionally, most pre-clinical studies dose prior to the onset of symptoms.

Therefore, there remains a need for a treatment for RTT and symptoms thereof.

### BRIEF SUMMARY OF THE INVENTION

The subject invention pertains to compositions comprising compounds that can alter short fatty acid Co-enzyme A (CoA) metabolism, including short fatty acid Co-A synthetase 3 (ACSS3), and methods of treating Rett Syndrome (RTT) and other neurodevelopmental and metabolic disorders, such as, for example, Fragile X and Leigh syndromes, comprising administering the subject compositions to a subject in need thereof. In preferred embodiments, the compound may comprise succinanilic acid or another compound as disclosed herein. The disclosed compounds can engage a majority of the identified targets and mechanisms, including, for example, altering the expression of genes encoding RUNX3, MeCP2, or proteins in the KEAP1-NFE2L2 pathway without inhibiting HDACs.

### BRIEF DESCRIPTION OF THE DRAWINGS

FIGS. 1A-1B. Pharmacokinetic data from plasma (FIG. 1A) and tissues (FIG. 1B), following oral dosing with either 50 mg/kg vorinostat or RVL002.

FIGS. 2A-2B. BioNav™ plots organized by chemical structure similarity of 40,000 compounds. Differential scores of predicted drugs mimicking vorinostat- or RVLOO2-dosed mouse brain (FIG. 2A) or femoral muscle (FIG. 2B) are plotted. Cluster of HDAC inhibitors is circled (FIG. 2B).

FIGS. 3A-3E. Venn diagrams showing the extend of similarities and differences in the genes and pathways significantly altered in mice treated with either vorinostat (50 mg/kg p.o.) or RVL002 (50 mg/kg p.o.) when compared to vehicle treated mice. Genes associated with network level changes in mice treated with vorinostat or RVL002 were analyzed and sorted according to Reactome.org pathways (91). RVL002 engages pathways in the brain and muscle tissues similar to those resulting from vorinostat dosing (FIG. 3A). Differential gene expression (DGE) analysis shows relatively even distributions of genes altered by either vorinostat or RVL002 in mice brain hemisphere sections and mice femoral muscle sections (FIG. 3B). Network probability analysis using BioNAV shows contrasting number of genes associated with network level changes between vorinostat or RVL002 (FIG. 3C). Vorinostat does not efficiently cross the blood brain barrier resulting in a lower modulation of network in brain tissues compared with muscle tissues. This is demonstrated by the identification of only a single HDAC associated pathway being modulated by vorinostat in the brain despite 97.3% overlap with RVL002 in muscle tissues (FIG. 3D). RVL002 engages MeCP2 pathways in the brain and muscle tissues (FIG. 3E).

FIGS. 4A-4F. FIG. 4A) Occupation density plot from tracking tadpoles swimming and resting locations over a 10-minute recording session (n=5 per 10 ml dish, at 30 frames per seconds), where high density means the tadpoles where recorded at that XY coordinate most frequently. FIG. 4A) wild-type tadpoles (WT) normal behavior is to swim along the edge of the dish in an area termed the “outer area”, while the area within the white center circle is termed the “inner area” and is mostly dark. FIG. 4B) RTT tadpoles (KD), a light shadow or footprint shaped like a tadpole can be seen suggesting a seizure event in the Rett model. Additionally, RTT tadpoles cover less distance in the outer area, preferring to cross the inner area. FIG. 4C) example of multiple parameters derived from the tracking system, highlighting metrics corresponding to behavioral phenotypes. FIG. 4D) one such metric of epoch-based entropy which captures information about the swimming behavior over a recording session and how a group diverges from the others, in this case indicating favorable improvement in RTT tadpole symptoms treated with RVL002 (epoch=15-second). FIG. 4E) one such metric is the inner area circular swimming. Locomotor assays is commonly used in mouse model of neurological diseases, it can also assess the behavior in tadpole models. Both vorinostat and RVL002 reduced the average inner dish circular swimming behavior in KD tadpoles compared to vehicle controls. FIG. 4F) viability measurement in WT and RTT tadpoles demonstrating that RVL002 is well tolerated and effective (percent from n=10 per group).

FIGS. 5A-5B. Mice (KO MeCP2^{-/y} or WT littermates) were treated with 50 mg/kg P.O. RVL002 daily following the onset of severe symptoms at 34 days post-natal (P34). RVL002 delayed the progression in severity of behavioral and physiological phenotypes associated with the loss of MeCP2 expression as measured by the Rett cumulative severity score (Guy et al., 2001, 2007) (FIG. 5A) and the optomotor assay (FIG. 5B).

FIGS. 6A-6C. Xenopus laevis tadpoles at 15 days post-fertilization (15 dpf) were recorded and their swimming behavior was analyzed to show that an in vivo Rett model also deficient in ACSS3 would respond differently to RVL002. Wild-type (WT), MeCP2 knockdown (mecp2-KD) and double knockdown of MeCP2 and ACSS3 (mecp2-acss3-2KD) tadpoles, were compared in the presence of absence of 50 μM RVL002 added to the swimming media every other day since 6 dpf. Mecp2-acss3-2KD tadpoles did not respond to RVL002 across exploratory behavior (FIG. 6A), swimming velocity (FIG. 6B) and the number of tadpoles in a school (FIG. 6C).

### DETAILED DISCLOSURE OF THE INVENTION

The present disclosure relates to compounds and compositions comprising succinanilic acid or other compounds and methods of treating Rett Syndrome (RTT) and other neurodevelopmental and metabolic disorders, such as, for example Fragile X and Leigh syndromes, or symptoms thereof or methods of treating a subject with a loss-of-function mutation of a gene encoding methyl-CpG binding protein (MeCP2) by administration of succinanilic acid or other compounds disclosed herein to a subject or in an in vitro setting.

The terms “treat”, “treating”, “inhibiting”, “inhibit”, “suppressing”, “suppress”, “decrease” and “decreasing” are used, in the context of this disclosure, to refer to the reduction, improvement, stabilization and/or elimination of a symptom of a disease, or slowing the progression of any disease of disorder disclosed within this application, for example, the treatment of RTT and normalizing expression levels of MeCP2. RTT and the other diseases or disorders disclosed herein can be treated by way of the administration of the compounds disclosed herein.

The term “therapeutically effective amount” is intended to constitute an amount of succinanilic acid or another compound disclosed herein that treats or suppresses RTT or another disease or disorder (as disclosed in this application) in a subject. In other words, succinanilic acid or a derivative thereof is administered in an amount sufficient to constitute a treatment of the disease or disorder, such as RTT, and cause a reduction, improvement, stabilization and/or elimination of a symptom of a disease or slowing the progression of the disease or disorder, such as a disease associated with the loss of function of MeCP2. Such amounts could range from about 0.01 to about 1,000 mg/kg of a subject to be treated. In other embodiments, the amounts administered can range between about 0.25 and about 5 mg/kg/day, between about 0.4 and 4.5 mg/kg/day, between about 0.6 and 4.25 mg/kg/day, between about 0.8 and 4 mg/kg/day, between about 1 and 3.75 mg/kg/day, between about 0.75 and 3 mg/kg/day, between about 0.5 and 2.75 mg/kg/day, or between about 0.45 and 2.5 mg/kg/day.

The terms “about” or “approximately” mean a range of ±0-20%, ±0 to 10%, or up to ±1% of a given value. In the context of compositions containing amounts of ingredients where the terms “about” or “approximately” are used, these compositions contain the stated amount of the ingredient with a variation (error range) of 0-10% around the value (X±10%). With respect to periods of time (days, weeks, months), the term is intended to include a period of ±6 hours for days, ±1 day for weeks, and ±7 days for months.

The term “and/of” is used, herein, as a function word to indicate that two words or expressions are to be taken together or individually. A₅ an example, the phrase “a, b, and/or c” be construed as a alone, b alone, c alone, a combination of a and b, a combination of a and c, a combination of b and c, or a combination of a, b, and c.

A₅ used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, include the phrases “consisting essentially of”, “consists essentially of”, “consisting”, and “consists” can be used interchangeably. The phrases “consisting essentially of” or “consists essentially of” indicate that the claim encompasses embodiments containing the specified materials or steps and those that do not materially affect the basic and novel characteristic(s) of the claim.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. When ranges are used herein (such as for dose ranges), specific embodiments of different combinations and subcombinations of these dose ranges (e.g., subranges within the disclosed ranges) are intended to be explicitly included.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art.

The active agents disclosed herein can be formulated into pharmaceutically acceptable compositions. Such compositions comprise one or more of the disclosed active agents in combination with a pharmaceutically acceptable carrier such as a phosphate buffered saline, a bicarbonate solution, or formulated with a carrier such as starch into a pill to produce a pharmaceutical composition. The carrier must be “acceptable” in the sense that it is compatible with the active ingredient (agent) of the composition, and preferably capable of stabilizing the active agent and not deleterious to the subject to be treated. The carrier is selected on the basis of the mode and route of administration and standard pharmaceutical practice. Suitable pharmaceutical carriers and diluents, as well as pharmaceutical necessities for their use, are described in Remington's Pharmaceutical Sciences.

A₅ used herein, “subject”, “host” or “organism” refers to any member of the phylum Chordata, more preferably any member of the subphylum vertebrata, or most preferably, any member of the class Mammalia, including, without limitation, humans and other primates, including non-human primates such as rhesus macaques, chimpanzees and other monkey and ape species; farm animals, such as cattle, sheep, pigs, goats and horses; domestic mammals, such as dogs and cats; laboratory animals, including amphibians (e.g., frogs, salamanders, caecilians), rabbits, mice, rats and guinea pigs; birds, including domestic, wild, and game birds, such as chickens, turkeys, ducks, and geese. The term does not denote a particular age or gender. Thus, adult, young, and newborn individuals are intended to be covered as well as male and female subjects. In some embodiments, a host cell is derived from a subject (e.g., tissue specific cells, such as hepatocytes).

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

A₅ used herein, a “pharmaceutical” refers to a compound manufactured for use as a medicinal and/or therapeutic drug.

In preferred embodiments, the compositions can comprise a chemical according to Formula (I):

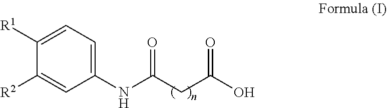

wherein R¹ is H

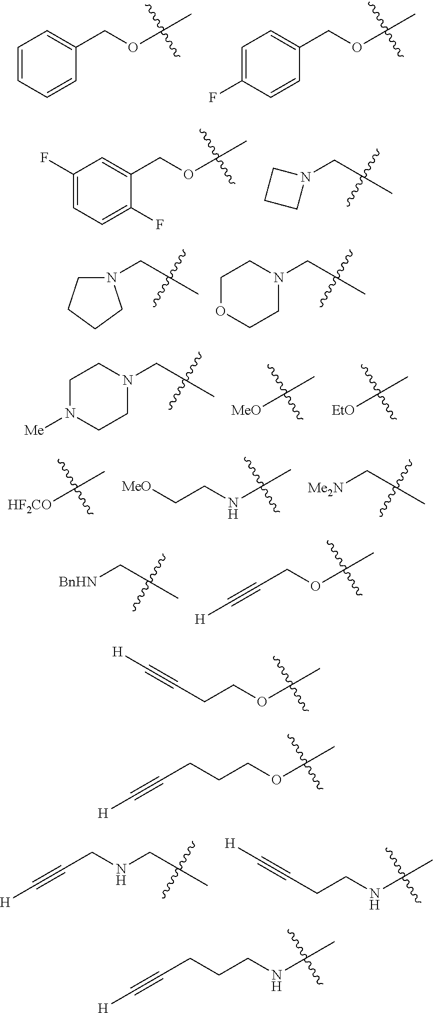

R² is H

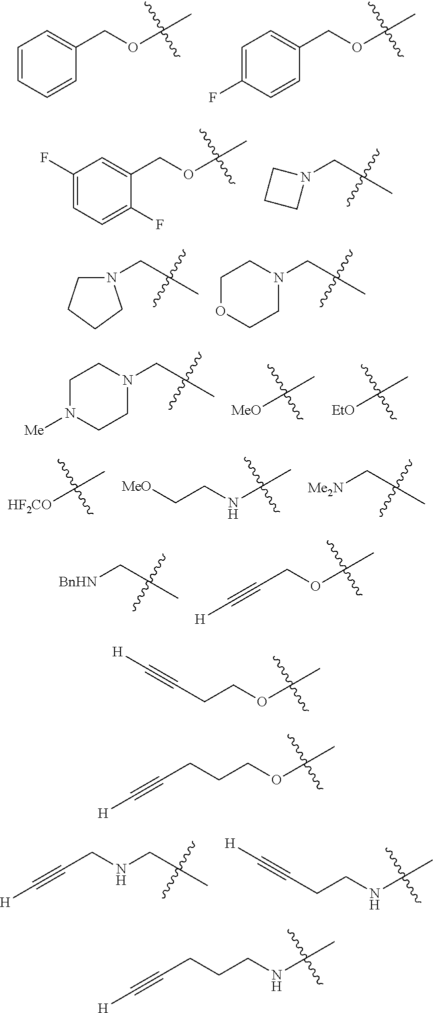

and n is an integer from 1 to 12. In certain embodiments, n is an integer from 1 to 4 or 6 to 12 when R¹ and R² are both H. In some embodiments, R¹ and R² can be the same or different. In other embodiments, R¹ is H and R² is any substituent other than H. In yet other embodiments, R² is H and R¹ is any substituent other than H.

In certain embodiments, the composition can comprise succinanilic acid, according to Formula (III:

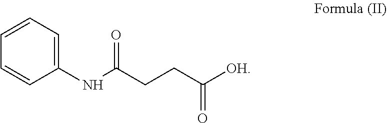 [SMILES: O=C(O)CCC(=O)Nc1ccccc1]

In certain embodiments, the compositions can comprise a compound according to Formula (IV) or Formula (V):

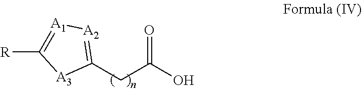

wherein R is H

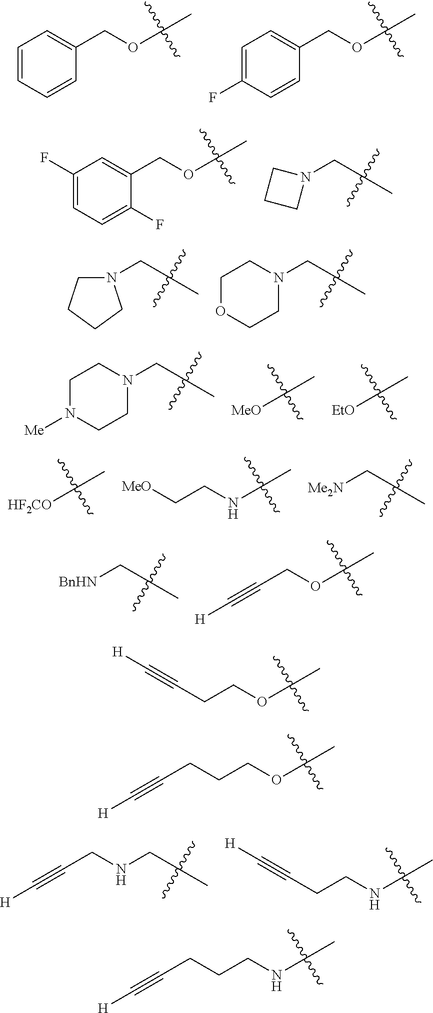

A₁ is N or CH;
A₂ is N or CH;
A₃ is O or S;
and n is an integer from 1 to 12;

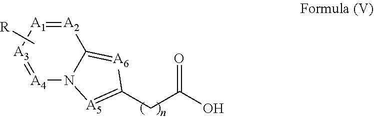

wherein R is H

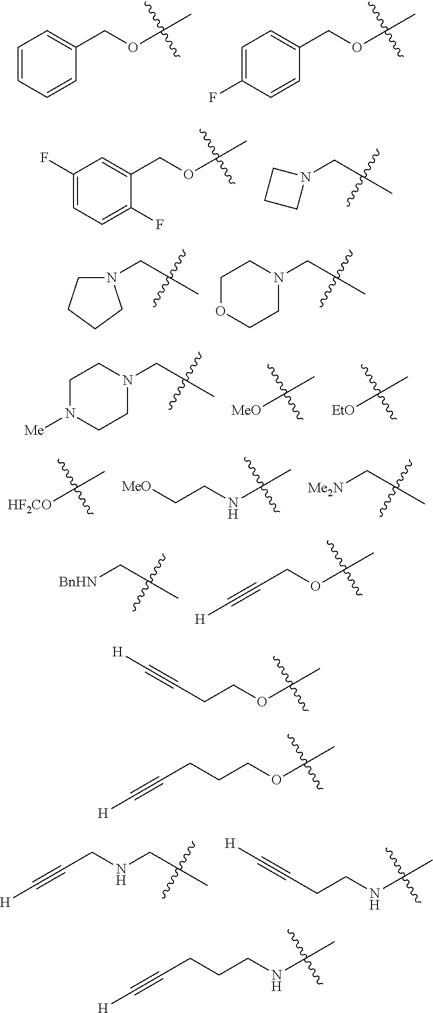

A₁ is N or CH;
A₂ is N or CH;
A₃ is N or CH;
A₄ is N or CH;
A₅ is N or CH;
A₆ is N or CH;
and n is an integer from 1 to 12. In some embodiments, A₁ through A₆ are CH. In other embodiments; A₅ is N and A₁₋₄ and A₆ are CH; A₆ is N and A₁₋₅ are CH; or A₄ and A₆ are N and A₁₋₃ and A₅ are CH.

In certain embodiments, the compositions can comprise a compound according to Formula (VI), Formula (VII), Formula (VIII), Formula (IX), Formula (X), or Formula (XI):

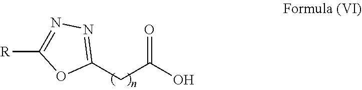

wherein R is H

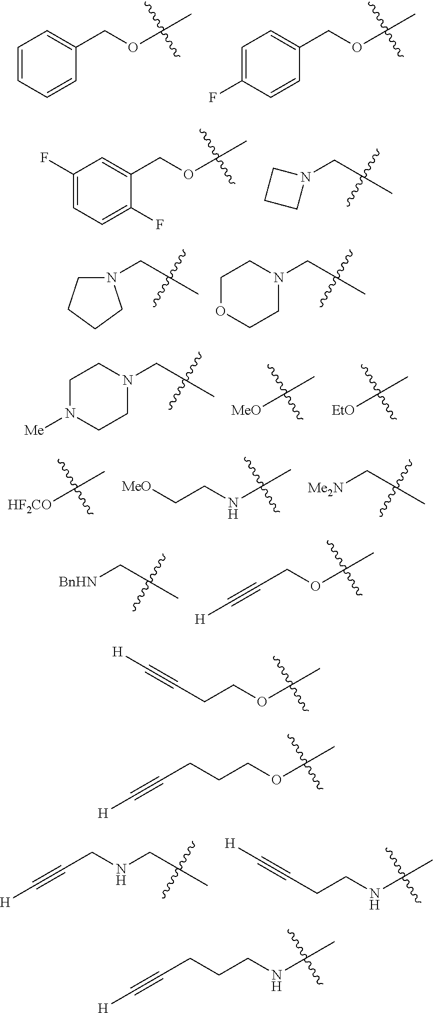

and n is an integer from 1 to 12;

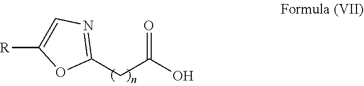

wherein R is H

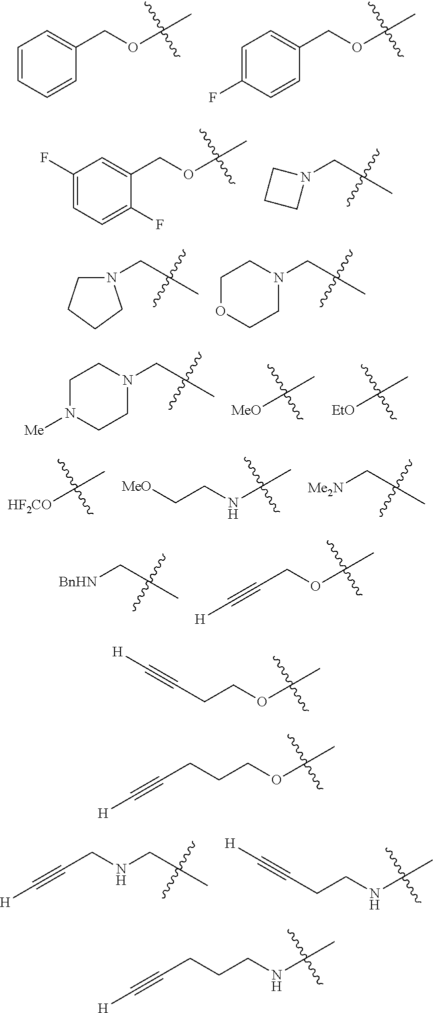

and n is an integer from 1 to 12;

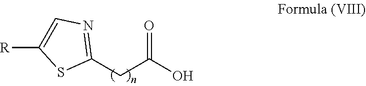

wherein R is H

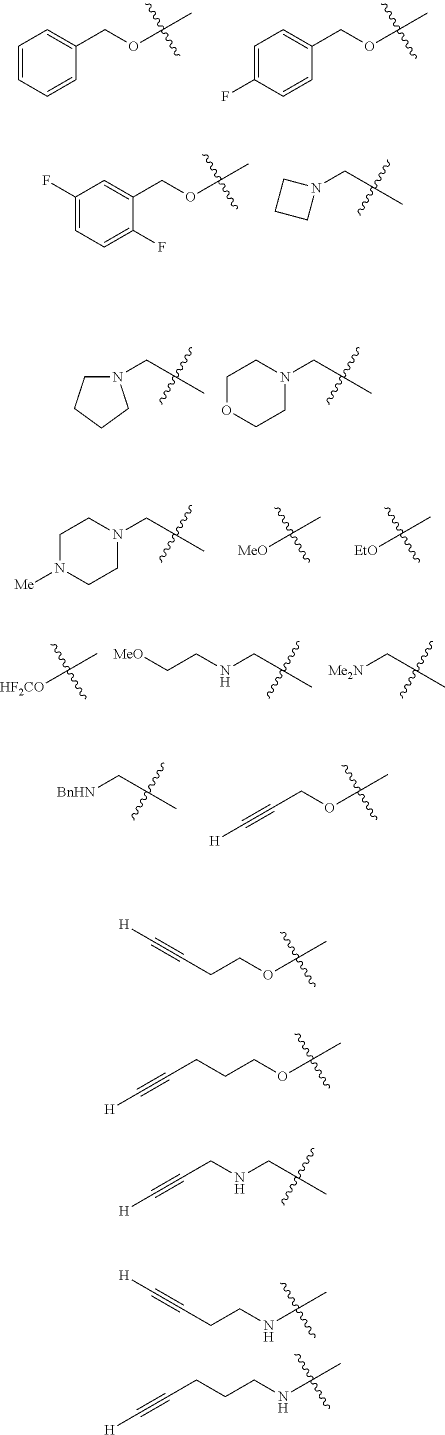

and n is an integer from 1 to 12;

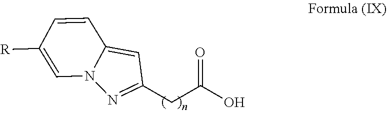

wherein R is H

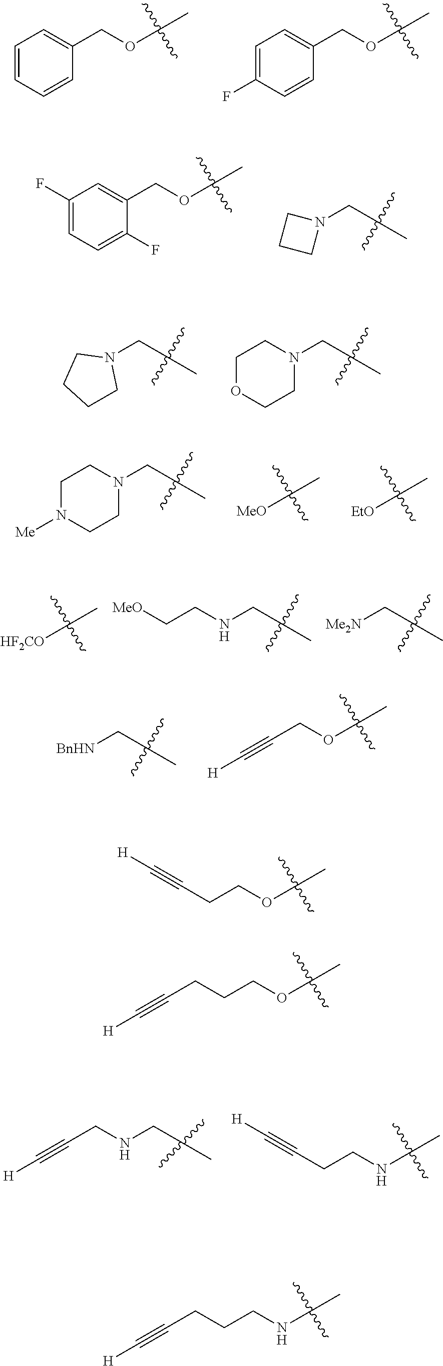

and n is an integer from 1 to 12;

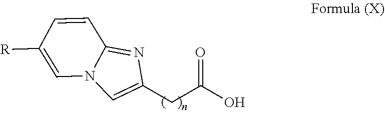

wherein R is H

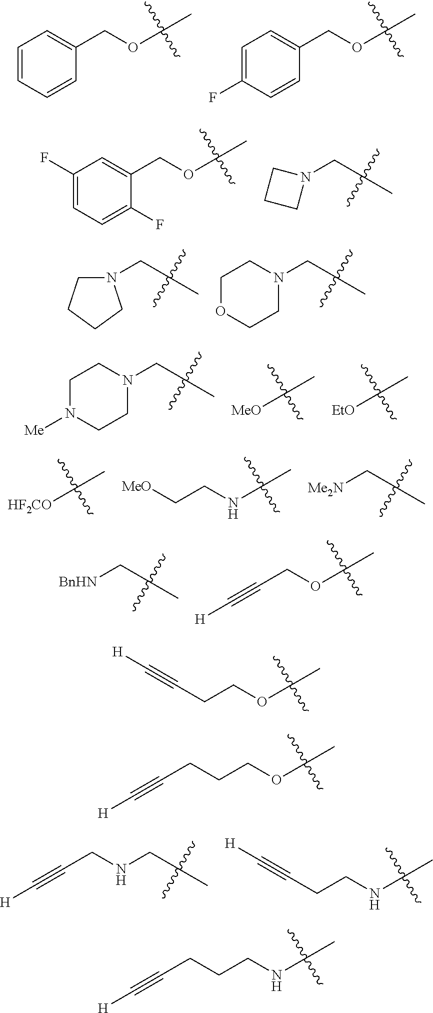

and n is an integer from 1 to 12;

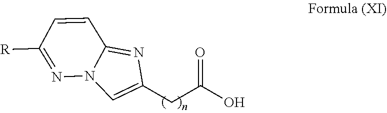

wherein R is H

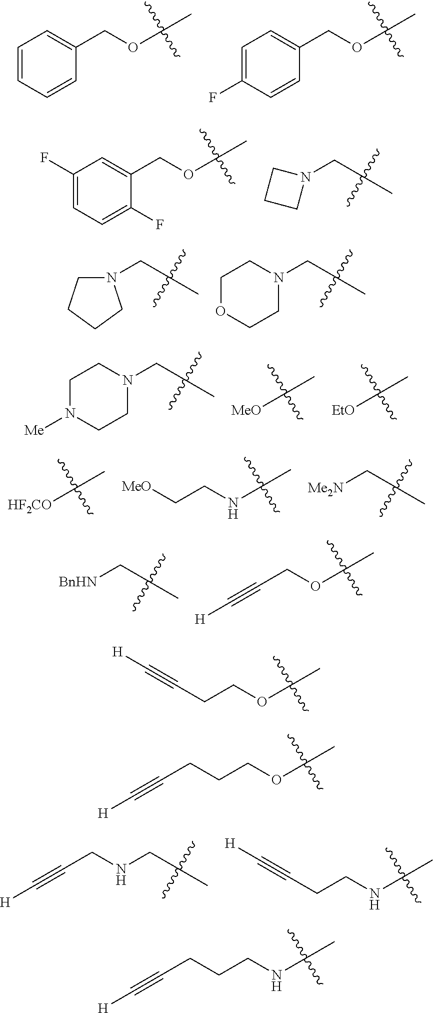

and n is an integer from 1 to 12.

In some embodiments, a compound or composition comprising one or more of the compounds according to Formulas (I) through (XI) can be administered to a subject. Non-limiting examples of such compositions include: succinanilic acid or a compound according to any one of Formulas (I) or (III)-(XI) that is to be administered to a subject and contains at least about 0.001%, about 0.01%, about 0.01%, about 0.1%, about 1%, about 5%, about 10%, about 15%, about 20%, about 25% succinanilic acid or a compound of Formula (I) by weight of the composition. In other embodiments, the composition contains between: about 0.001% and about 25% succinanilic acid or a compound of Formula (I) or (III)-(XI), about 0.001% and about 20% succinanilic acid or a compound of Formula (I) or (III)-(XI), about 0.001% and about 15% succinanilic acid or a compound of Formula (I) or (III)-(XI), about 0.001% and about 10% succinanilic acid or a compound of Formula (I) or (III)-(XI), about 0.001% and about 5% succinanilic acid or a compound of Formula (I) or (III)-(XI), about 0.100% and about 2.5% succinanilic acid or a compound of Formula (I) or (III)-(XI), about 0.100% and about 2.0% succinanilic acid or a compound of Formula (I) or (III)-(XI), about 0.100% and about 1.5% succinanilic acid or a compound of Formula (I) or (III)-(XI), about 0.100% and about 1.0% succinanilic acid or a compound of Formula (I) or (III)-(XI), about 0.100% and about 0.5% succinanilic acid or a compound of Formula (I) or (III)-(XI), about 0.100% and about 25% succinanilic acid or a compound of Formula (I) or (III)-(XI), about 0.100% and about 20% succinanilic acid or a compound of Formula (I) or (III)-(XI), about 0.100% and about 15% succinanilic acid, about 0.100% and about 10% succinanilic acid, about 0.100% and about 5% succinanilic acid, about 0.500% and about 25% succinanilic acid or a compound of Formula (I) or (III)-(XI), about 0.500% and about 20% succinanilic acid or a compound of Formula (I) or (III)-(XI), about 0.500% and about 15% succinanilic acid, about 0.500% and about 10% succinanilic acid, about 0.500% and about 5% succinanilic acid or a compound of Formula (I) or (III)-(XI), about 1% and about 5% succinanilic acid or a compound of any one of Formulas (I) or (III)-(XI) by weight of the composition. In these embodiments the remainder of the composition comprises fillers or other solid pharmaceutically acceptable excipients. As would be apparent to those skilled in the art, any combination of compounds according to Formula (I) through Formula (XI) can be used in the preparation of a composition as disclosed herein. In other embodiments, the compounds are administered in amounts that range from about 0.01 to about 1,000 mg/kg of a subject to be treated. In other embodiments, the amounts administered can range between about 0.25 and about 5 mg/kg/day, between about 0.4 and 4.5 mg/kg/day, between about 0.6 and 4.25 mg/kg/day, between about 0.8 and 4 mg/kg/day, between about 1 and 3.75 mg/kg/day, between about 0.75 and 3 mg/kg/day, between about 0.5 and 2.75 mg/kg/day, or between about 0.45 and 2.5 mg/kg/day. Where combinations of compounds are used, the amount of active ingredient (one or more compound of Formulas (I) through (XI)) are present in the amounts described within this paragraph.

The compounds and compositions disclosed herein can be administered by any acceptable route for the treatment of any disease disclosed herein. For example, the treatment of RTT or a subject with a mutation of a gene encoding MeCP2 that confers a loss-of-function of MeCP2. Non-limiting examples include administering the compounds and/or compositions disclosed herein orally, by injection, by subcutaneous injection, by intraperitoneal injection, by intravenous infusion, topically, or by inhalation.

The subject compounds can be formulated according to known methods for preparing pharmaceutically useful compositions. In general, the compositions of the subject invention will be formulated such that an effective amount of the subject compounds is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject compounds include, but are not limited to, water, saline, oils including mineral oil, ethanol, dimethyl sulfoxide, gelatin, cyclodextrans, magnesium stearate, sodium croscarmellose, dextrose, cellulose, sugars, calcium carbonate, glycerol, alumina, starch, and equivalent carriers and diluents, or mixtures of any of these. The subject composition can further comprise one or more pharmaceutically acceptable carriers and/or excipients. The term “pharmaceutically acceptable” as used herein means compatible with the other ingredients of a pharmaceutical composition and not deleterious to the recipient thereof. Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target proteins or with the composition, carrier or excipient use in the subject compositions may be contemplated.

In one embodiment, the compounds of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

To provide for the administration of such dosages for the desired therapeutic treatment, pharmaceutical compositions of the invention will advantageously comprise between about 0.1% and 99%, and especially, 1% and 15% by weight of the total of the subject compounds based on the weight of the total composition including carrier or diluent.

The subject compounds can also be administered utilizing liposome technology, slow release capsules, implantable pumps, and biodegradable containers. These delivery methods can, advantageously, provide a uniform dosage over an extended period of time.

The subject invention also concerns a packaged dosage formulation comprising in one or more packages, packets, or containers the subject compounds and/or a composition of the subject invention formulated in a pharmaceutically acceptable dosage. The package can contain discrete quantities of the dosage formulation, such as tablet, capsules, lozenge, and powders. The quantity of the subject compounds in a dosage formulation and that can be administered to a patient can vary from about 1 mg to about 1000 mg. In some embodiments, the amount is in the range of 5 mg to 500 mg, to be administered 1, 2, 3, or 4 times per day, for about 2, about 3, about 4, about 5, about 6, about 7, about 10, about 14, about 21, about 28, about 35, about 40, about 45, about 50, about 55, about 60, about 120, about 180, about 365 days or more days. In some embodiments, the subject compounds or compositions can be administered a certain number of times per week or month (for example, 1, 2, 3, 4, 5, 6, or 7 times per week or between 1 day and x days per month, where x is 28, 29, 30, or 31 days depending on the month or year (if a leap year)). “A week” refers to a period of time of about 5, about 6 or about 7 days. “A month” refers to a period of time of about 28, about 29, about 30, or about 31 days.

As discussed above, therapeutically effective amounts of the subject compositions are administered to a subject in need of treatment of RTT. Thus, the subject compounds can be administered in an amount that ranges from about 0.01 mg/kg to about 150 mg/kg of a subject to be treated; about 1 mg/kg to about 75 mg/kg of a subject to be treated; about 1 mg/kg to about 50 mg/kg of a subject to be treated; about 10 mg/kg to about 100 mg/kg of a subject to be treated; about 10 mg/kg to about 75 mg/kg of a subject to be treated; about 10 mg/kg to about 50 mg/kg of a subject to be treated; about 1 mg/kg to about 75 mg/kg of a subject to be treated; about 10 mg/kg to about 30 mg/kg of a subject to be treated; about 15 mg/kg to about 30 mg/kg of a subject to be treated; about 15 mg/kg to about 25 mg/kg of a subject to be treated; or in an amount of about 20 mg/kg of a subject to be treated. In other embodiments, the compounds can be administered in amounts that range from about 0.01 to about 1,000 mg/kg of a subject to be treated. In other embodiments, the amounts administered can range between about 0.25 and about 5 mg/kg/day, between about 0.4 and 4.5 mg/kg/day, between about 0.6 and 4.25 mg/kg/day, between about 0.8 and 4 mg/kg/day, between about 1 and 3.75 mg/kg/day, between about 0.75 and 3 mg/kg/day, between about 0.5 and 2.75 mg/kg/day, or between about 0.45 and 2.5 mg/kg/day.

In certain embodiments, the subject compositions or compounds can be administered before symptoms of RTT have developed, including, for example, impairments in language and coordination, repetitive movements, slower growth, or a smaller head, or before a subject is diagnosed with RTT. Additionally or alternatively, the subject compositions can be administered after symptoms of RTT have developed or after a subject has been diagnosed with RTT. In certain embodiments, diagnosing RTT comprises identifying at least one mutation in the gene encoding MeCP2. In some embodiments, the compounds can be administered in amounts that range from about 0.01 to about 1,000 mg/kg of a subject to be treated. In other embodiments, the amounts administered can range between about 0.25 and about 5 mg/kg/day, between about 0.4 and 4.5 mg/kg/day, between about 0.6 and 4.25 mg/kg/day, between about 0.8 and 4 mg/kg/day, between about 1 and 3.75 mg/kg/day, between about 0.75 and 3 mg/kg/day, between about 0.5 and 2.75 mg/kg/day, or between about 0.45 and 2.5 mg/kg/day.

In certain embodiments, the administration of the subject compounds or compositions can modulate the expression of genes encoding RUNX3, MeCP2, or proteins in the KEAP1-NFE2L2 pathway. In certain embodiments, the expression of the gene encoding MeCP2 is increased. In certain embodiments, the administration of the subject compounds or compositions alters the signaling pathways controlling α- and β-tubulins, acetyl CoA synthetase 3, and ribosome protein S6. In certain embodiments, the administration of the subject compounds or compositions normalizes signaling pathways involved in deubiquitinating, unwinding of DNA, regulation of HMOXI expression and activity, displacement of DNA glycosylase by APEX1, G2/M checkpoints, SCF-beta-TrCP mediated degradation of Emil.

In yet other embodiments, compounds and compositions disclosed herein are administered in therapeutically effective amounts for the treatment of various diseases and disorders selected from the group consisting of fatty liver/NAFLD/NASH/MASH steatosis, fibrosis, and inflammation states; obesity; other dystonias such as DM1 or KMT2B; Parkinson's disease; Pitt Hopkins syndrome; Leigh syndrome, including mutations in MT-ND1, SURF1, or other Complex IV genes; MEF2C haploinsufficiency; CIC haploinsufficiency; Fragile X syndrome; AP-4 neurodevelopmental disorders, FOXG1 syndrome; CDKL5 Deficiency Disorder; disorders caused by a deletion or mutation of the SHANK3 gene, including Phelan-McDermid syndrome and other Rett-like disorders. The disclosed compounds and/or compositions can be used alone or in combination with other treatments for the disclosed diseases and disorders. Thus, where the disclosed compounds and/or compositions are administered to a subject in combination with other therapeutic agents the combination treatment can be administered to the subject as a combined composition, simultaneously as separate compositions or sequentially as separate compositions. Sequential administration contemplates the temporally separated administration of the distinct therapeutic agents. One non limiting example provides for the administration of a first therapeutic agent (e.g., a disclosed compound and or composition) to the subject and a second therapeutic agent that is administered within about 1 minute to about 72 hours after the administration of the first therapeutic agent for the treatment of a given disease or disorder.

Accordingly, one embodiment provides methods of treating NASH comprising the administration of a compound or composition disclosed herein alone or in combination with one or more therapeutic agent selected from the group consisting of: (a) elafibranor in an amount of about 50 mg/d to about 120 mg/d; (b) saroglitazar in an amount of about 4 mg/d to about 20 mg/d; (c) obeticholic acid in an amount of about 10 mg/d to about 25 mg/d; (d) tropifexor in an amount of about 2 mg/d to about 20 mg/d; (e) aramchol in an amount of about 400 mg to about 600 mg; (f) selonsertib in an amount of about 6 mg/d to about 40 mg/d alone or in combination with simtuzumab in an amount of about 125 mg per week or every two weeks; (g) selonsertib in an amount of about 6 mg/d to about 40 mg/d alone or in combination with fenofibrate in an amount of about 30 mg/d to about 200 mg/d; (h) cenicriviroc in an amount of about 75 mg/day to about 150 mg/d; (i) emricasan in an amount of about 5 mg/BID to about 50 mg/BID; (j) simtuzumab in an amount of about 75 mg to about 700 mg per week or every two weeks or an amount of about 75 mg to about 200 mg per week or every two weeks; (k) galactoarabino rhamnogalacturonate in an amount of about 2 mg/d to 10 mg/kg lean body mass every other week; (1) pioglitazone in an amount of about 30 mg/d alone or in combination with vitamin E in an amount of about 800 IU/d; (m) pioglitazone in an amount of about 45 mg/d; (n) aramchol in an amount of about 100 mg/d to about 300 mg/d; (o) liraglutide in an amount of about 1.8 mg/d; (p) pegylated FGF21 in an amount of about 5 mg/d to about 50 mg/d, administered subcutaneously; (q) EDP-305 (CAS Reg. No. 1933507-63-1 and disclosed in U.S. Pat. No. 10,208,081 in Example 3 or 77, which are hereby incorporated by reference in their entirety) in an amount of about 5 mg/d to about 50 mg/d; (r) encapsulated hyperimmune bovine colostrum enriched with anti-LPS antibodies in an amount of about 300 mg to about 1500 mg up to three times daily; (s) lanifibranor in an amount of about 200 mg/d to about 1200 mg/d or about 400 mg/d; (t) LMB763 in an amount of about 5 mg/d to about 750 mg/d; (u) licogliflozin in an amount of about 100 mg/d to about 300 mg/d; (v) MGL-3196 (resmetirom) in an amount of about 60 mg/d to about 100 mg/d; (w) MSDC 0602K (Azerniglitazone potassium) in an amount of about 50 mg/d to about 300 mg/d; (x) PF-05221304 (clesacostat) in an amount of about 2 mg/d to about 60 mg/d; (y) SAR425899 (bamadutide) in an amount of about 20 microgram to about 300 microgram administered once daily by subcutaneous injection; (z) selonsertib+GS-0976 (firsocostat)+GS-9674 (cilofexor) in an amount of about 15 mg/d to about 30 mg/d for each active agent; (aa) semaglutide in an amount of about 0.1 mg to about 0.5 mg administered subcutaneously once daily; (bb) volixibat in an amount of about 5 mg/d to about 50 mg/d; and (cc) a statin agent such as atorvastatin in an amount of about 10 mg/d to about 80 ng/d, fluvastatin in an amount of about 2.0 mg/d to about 80 mg/d, rosuvatatin in an amount of about 5 mg/d to about 40 mg/d, pravastatin in an amount of about 10 mg/d to 80 mg/d, sinvastatin in an amount of about 5 rng/d to 40 mg/d, lovastatin in an amount of about 10 mg/d to 80 mg/d, or pitavastatin in an amount of about 1 mg/d to 4 mg/d.

Accordingly, one embodiment provides methods of treating NASH/Cholestasis/NAFLD comprising the administration of a compound or composition disclosed herein alone or in combination with one or more therapeutic agent selected from the group consisting of: (a) vitamin E; (b) pioglitazone; (c) obeticholic acid; (d) tropifexor; (e) FGF-19; (f) elafibranor; (g) lanifibranor; (h) FGF-21; (i) dimtuxumab; and combinations thereof.

Accordingly, one embodiment provides methods of treating obesity comprising the administration of a compound or composition disclosed herein alone or in combination with one or more therapeutic agent selected from the group consisting of: (a) bupropion—naltrexone; (b) liraglutide; (c) orlistat; (d) phentermine-topiramate; (e) semaglutide; (f) setmelanotide; (g) tirzepatide and combinations thereof.

Another embodiment provides methods of treating Parkinson's disease comprising the administration of a compound or composition disclosed herein alone or in combination with one or more therapeutic agent selected from the group consisting of: (a) carbidopa-levodopa; (b) dopamine agonists, e.g., pramipexole, rotigotine, apomorphine; (c) monoamine oxidase B (MAO B) inhibitors, e.g., selegiline, rasagiline, safinamide; (d) catechol O-methyltransferase (COMT) inhibitors, e.g., entacapone, opicapone, tolcapone; (e) anticholinergics, e.g., benztropine, trihexyphenidyl; (f) amantadine; (g) adenosine receptor antagonists (A2A antagonists), e.g., istradefylline; (h) pimavanserin; and (i) deep brain stimulation.

Another embodiment provides methods of treating Leigh syndrome comprising the administration of a compound or composition disclosed herein alone or in combination with one or more therapeutic agent selected from the group consisting of: (a) citric acid (sodium citrate) or sodium bicarbonate; (b) thiamine (vitamin B1); (c) antiepileptic/anticonvulsant drugs such as carbamazepine, clobazam, clonazepam, eslicarbazepine acetate, felbamate, gabapentin, lacosamide, lamotrigine, levetiracetam, oxcarbazepine, phenobarbital, phenytoin, pregabalin, primidone, rufinamide, stiripentol, tiagabine, topiramate, vigabatrin, zonisamide, and combinations thereof.

Another embodiment provides methods of treating Fragile X comprising the administration of a compound or composition disclosed herein alone or in combination with one or more therapeutic agent selected from the group consisting of: (a) lithium carbonate; (b) gabapentin; (c) risperidone; (d) olanzapine; (e) carbamazepine; (f) lamotrigine; (g) methylphenidate and dextroamphetamine; (h) venlafaxine and nefazodone; (i) fluoxetine; (j) sertraline and citalopram and escitalopram; (k) trazodone; (1) melatonin; (m) recombinant Dgkk gene for Fragile X syndrome gene therapy (disclosed in U.S. Pat. No. 11,597,936, which is hereby incorporated by reference in its entirety).

Accordingly, one embodiment provides methods of treating Rett syndrome comprising the administration of a compound or composition disclosed herein alone or in combination with one or more therapeutic agent selected from the group consisting of: (a) such as trofinetide; (b) anticonvulsant or anti-seizure drugs such as valproic acid, cannabidiol, and levetiracetam. In some embodiments, the compound disclosed herein can be administered in amounts that range from about 0.01 to about 1,000 mg/kg of a subject to be treated. In other embodiments, the amounts administered can range between about 0.25 and about 5 mg/kg/day, between about 0.4 and 4.5 mg/kg/day, between about 0.6 and 4.25 mg/kg/day, between about 0.8 and 4 mg/kg/day, between about 1 and 3.75 mg/kg/day, between about 0.75 and 3 mg/kg/day, between about 0.5 and 2.75 mg/kg/day, or between about 0.45 and 2.5 mg/kg/day.

Another embodiment provides methods of treating disorders caused by a deletion or mutation of the SHANK3 gene, including Phelan-McDermid syndrome comprising amounts could range from about 0.01 to about 1,000 mg/kg of a subject to be treated. In other embodiments, the amounts administered can range between about 0.25 and about 5 mg/kg/day, between about 0.4 and 4.5 mg/kg/day, between about 0.6 and 4.25 mg/kg/day, between about 0.8 and 4 mg/kg/day, between about 1 and 3.75 mg/kg/day, between about 0.75 and 3 mg/kg/day, between about 0.5 and 2.75 mg/kg/day, or between about 0.45 and 2.5 mg/kg/day the administration of a compound or composition disclosed herein alone or in combination with one or more therapeutic agent selected from the group consisting of: (a) antiepileptic/anticonvulsant drugs such as carbamazepine, clobazam, clonazepam, eslicarbazepine acetate, felbamate, gabapentin, lacosamide, lamotrigine, levetiracetam, oxcarbazepine, phenobarbital, phenytoin, pregabalin, primidone, rufinamide, stiripentol, tiagabine, topiramate, vigabatrin, zonisamide (b) ithium; (c) risperidone; (d) gabapentin; (e) olanzapine; (f) lamotrigine; (g) methylphenidate and dextroamphetamine; (h) venlafaxine and nefazodone; (i) fluoxetine; (j) sertraline and citalopram and escitalopram; (k) trazodone; and (1) melatonin.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1-Use of Vorinostat to Treat RTT

Rare NDDs, such as RTT, have eluded the development of effective therapeutics due to the lack of in vitro screens directly associated with the cognitive and behavioral presentation of the disease. In addition, the far-ranging impact of methyl-CpG binding protein (MeCP2) as a central transcriptional regulator complicates target identification using bioinformatics alone. BioNav™ is an integrated platform using computational prediction to find effective treatments for NDDs, starting with RTT. The drug prediction algorithm uses a probabilistic network model to predict screening candidates from a library of more than 40,000 biologically active small molecules, based on a compound's ability to revert the entire disease network state back to health. We use CRISPR to create mosaic knockouts in embryos, which develop into a tadpole population presenting a range of gene expression knockdown (KD) across the organism and lead to a range of disease phenotypes and severities. This genetically diverse tadpole population of hundreds of individuals presents a spectrum of symptoms and severities, like the patient population.

Briefly, Xenopus embryos at the 4-cell stage were microinjected with Cas9 protein and guide RNA complex aimed at 3 sites of the MeCP2 gene corresponding to DNA binding and the mutation locations of the most severe patient cases. Embryos could develop into free-swimming tadpoles, and no effect on viability was observed relative to controls. Tadpoles presented a spectrum of behavioral phenotypes, including seizures. qPCR analysis confirmed a range of MeCP2 KO of 5-95% of the cells, and immunostaining of tissue sections indicated mosaic and overall reduction of MeCP2 protein levels and a-tubulin acetylation (Novak et al. 2022). Based on seizure and behavioral screens in RTT tadpoles, we evaluate the tolerability and efficacy of any number of predicted compounds. The top performing drug is vorinostat, a hydroxamic acid pan-Histone Deacetylase inhibitor (HDACi), approved for cutaneous T cell lymphoma (Novak et al., 2022). Using the methyl-CpG binding protein (MeCP2)-null mouse model (KO), we observed effective improvement in older mice presenting with extensive symptoms prior to treatment according to the Rett cumulative severity score, in which each mouse is given a scored from 0-2, with 0 representing the absence of symptoms and 2 representing the severe manifestation of symptoms, for each of six phenotypes: tremor, gait abnormalities, immobility, hind-limb clasping, breathing, and general condition. These six scores are then summed to provide a cumulative severity score from 0-12 (Guy et al., 2001, 2007). Performing a head-to-head comparison, trofinetide (Acadia Pharmaceuticals), which showed positive results in its Phase 3 trial, did not improve the cumulative severity score in the MeCP2-null mouse model (Novak et al., 2022).

### Example 2-Computational Drug Prediction and Drug Efficacy in the Rtt Tadpole

RTT tadpole transcriptomes were compared against wild-type (WT) sibling transcriptomes using our proprietary BioNav™ software, which ranked the entirety of compounds from the National Institute of Health (NIH) Library of Integrated Network-Based Cellular Signatures (LINCS) database (Keenan et al., 2018) that were predicted to shift a RTT state to a healthy state. The top candidates were downselected to include only FDA-approved drugs and screened in the RTT tadpole model using behavior, survival, and seizures as an efficacy readout, together with toxicity phenotypes. Of the top 6 drugs, 2 (vorinostat and ivermectin) showed stronger reversal of RTT symptoms at nontoxic concentrations. Trofinetide was used to evaluate the translational capability of the Xenopus RTT KD model, and it showed efficacy, albeit lower than the predicted drugs. Vorinostat was selected for more detailed evaluation in tadpoles and mouse models. MeCP2 deficit resulted in abnormal cilia morphology in the olfactory bulb (Novak et al., 2022) which was reversed with vorinostat administration. Interestingly, a-tubulin acetylation increased in the brain of RTT models but decreased elsewhere in the body and was normalized by vorinostat in all cases. Ciliary morphology was also normalized by drug treatment across the entire body, and vorinostat reduced Ib4+ inflammatory cells in the GI tract (Novak et al., 2022). These results suggest a novel and HDAC-independent mechanism for vorinostat.

### Example 3-Evaluation of Vorinostat in the MeCP2-Null Mouse Model (B6.129P2(C)-MECP2^{TM1.1BIRD}/J

Using trofinetide as a positive control, we evaluated the feasibility of initiating drug dosing starting at 30 days post-natal (P30), unlike the majority of RTT pre-clinical studies starting before the onset of symptoms. We proceeded to dose Mecp2-null male mice and WT littermates with vorinostat (50 and 100 mg/kg) or trofinetide (75 mg/kg) dosed daily via intraperitoneal (IP) injection. Vorinostat demonstrated better efficacy at 50 mg/kg using the Rett cumulative severity scoring system (Guy et al., 2001, 2007). Vorinostat also outperformed trofinetide in Y-maze performance, brain and GI tubulin acetylation normalization, reduction of inflammatory cytokine levels and reduction of goblet cells inflammation in the colon. Subsequently, pharmacokinetic (PK) assay indicated that oral dosing would deliver a more sustained release of vorinostat at lower concentrations. We evaluated the comparative efficacy of oral vs. IP dosing of vorinostat in MeCP2-null mice starting at 36 days post-natal (P36), well after the onset of symptoms, to better mimic clinical presentation of RTT patients. We observed oral vorinostat to have improved efficacy when starting treatment after the onset of symptoms, while IP delivery and trofinetide failed to show efficacy (Novak et al., 2022). Histological evaluation suggests that the response to vorinostat is broad and targets both the central nervous system (CNS) and somatic phenotypes associated with RTT, including microglia, inflammation, colon, lung and muscles (Novak et al., 2022).

### Example 4-Therapeutic Mechanism and Drug Target Discovery

We evaluated the therapeutic mechanism involved through preliminary application of our multi-omics BioNav™ workflow. By modifying the in vitro Thermal Proteome Profiling method (Franken et al. 2015) to whole cold-blooded organisms, we were able to investigate the protein-drug interactions of vorinostat and ivermectin. We identified HDAC family members for vorinostat, but we found an additional 17 non-HDAC targets relating to cytoskeletal components, post-translational acetylation, and acetyl metabolism, suggesting normalization of acetylation plays a widespread role in therapeutic mechanism. α- and β-tubulins, acetyl CoA synthetase 3, and ribosome protein S6 were significant, direct targets of both drugs (Novak et al., 2022). Given our observations of tubulin hyper- and hypo-acetylation, depending on the tissue type, due to MeCP2 knockdown (KD), we evaluated these targets further. In microtubule polymerization assays, both vorinostat and ivermectin demonstrated mild microtubule polymerization effects. We have identified similarities between the chemical structure of vorinostat and a known acetyl CoA synthetase inhibitor, further supporting the rationale for the proposed aims to validate discovered targets and develop RTT-specific therapeutics.

HDACi have been proposed to treat mood, neurodegenerative, and NDDs, including RTT (M. Shahbazian et al., 2002; Abel and Zukin, 2008; Hauke et al., 2009; Fischer et al., 2010; Benito et al., 2015; Mahgoub et al., 2016; Citraro et al., 2017; Subramanian et al., 2017; Cao, Zwinderman and Dekker, 2018; Landucci et al., 2018; Singh et al., 2018; Basu et al., 2019; Li et al., 2019; Petrozziello et al., 2020; Napoletani et al., 2021; Shen et al., 2021). MeCP2 is known to associate with and be regulated by some of the HDACs (Nan et al., 1998; M. Shahbazian et al., 2002; Mahgoub et al., 2016; Landucci et al., 2018; Ehrhart et al., 2020; Napoletani et al., 2021).

The polypharmacologic or off-target aspects of HDACi could explain why we and others have observed improvements in pre-clinical models of neurological disorders. HDAC6, one of the known targets of vorinostat, can deacetylate a-tubulin. However, we observed that tissue-specific a-tubulin hypo- and hyper-acetylation in MeCP2-deficient models were being normalized by vorinostat (Novak et al., 2022). The therapeutic mechanism for RTT could be indirect or HDAC-independent. HDACi are associated with adverse side effects, including fatigue, nausea, vomiting, and cardiotoxicity. Therefore, identifying a non-HDACi with similar efficacy as Vorinostat would lead to disease-specific targets and mechanisms with clinical benefits and fewer side effects.

### Example 5-Evaluation of Succinanilic Acid (Rvl002) for Treating Rett Syndrome

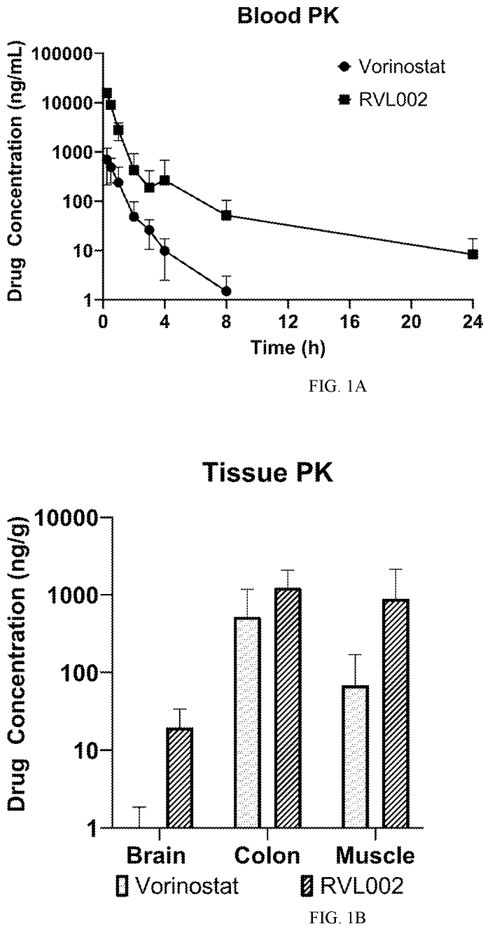

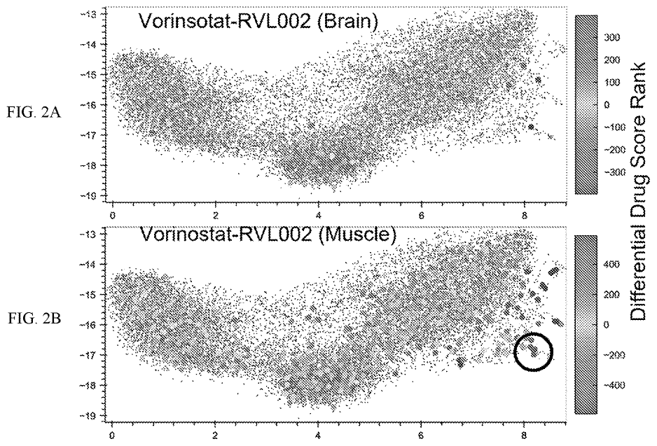

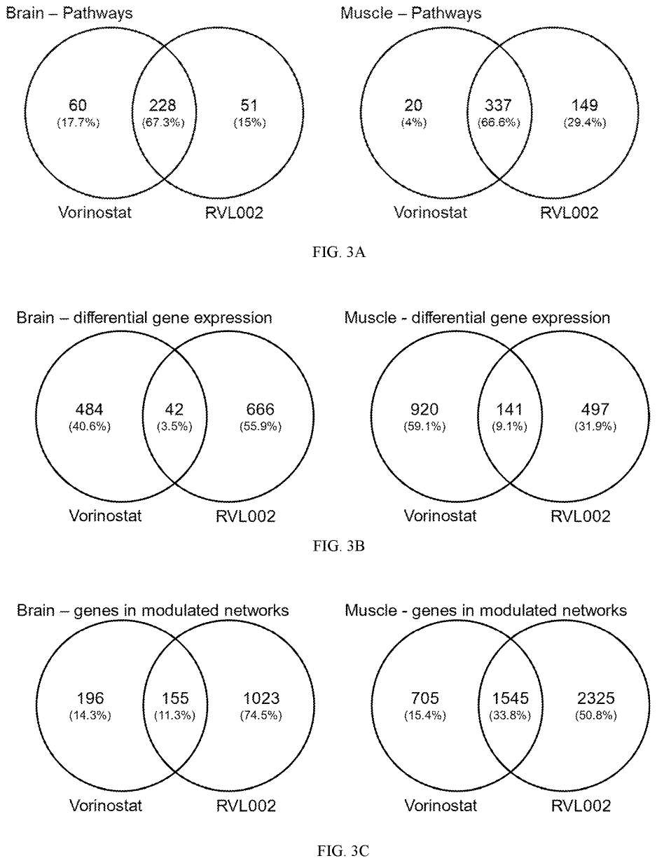

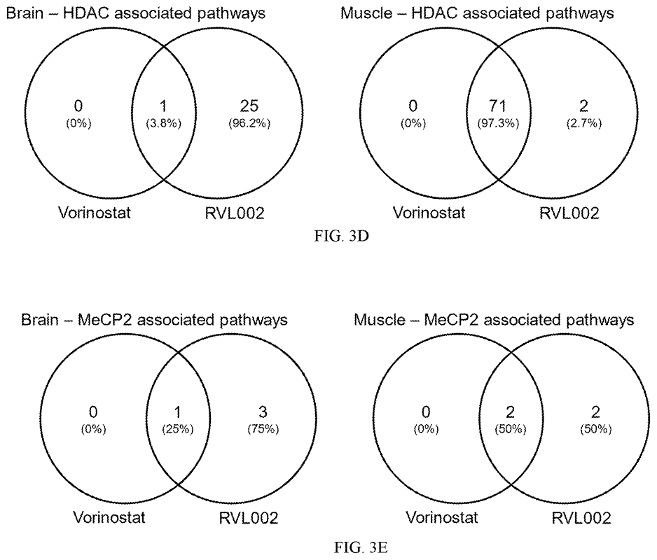

The newly identified protein targets for vorinostat, led to the discovery of a new chemical compound, RVL002, which we found offers comparable transcriptional changes as vorinostat does but with improved pharmacokinetic and pharmacodynamic (PK/PD) properties. The PK values show that RVL002 reaches higher peak plasma concentration for a longer period of time (Table 1), and show better biodistribution including in crossing the blood brain barrier (FIGS. 1A-1B). Brain and muscle tissues from these mice were submitted to RNA sequencing (RNAseq) and analyzed on our BioNav™ generated from the network model of 40,000 compounds with transcriptomic data. Brain RNAseq data showed minimal difference between vorinostat and RVL002 exposures, presumably due to the chemical similarity of the two compounds (FIG. 2A), while muscle showed greater differences, notably in the cluster of HDAC inhibitors (circled in FIG. 2B) demonstrating that RVL002 acts through non-HDAC-mediated pathways. By taking our calculated interacting gene sets we identified genes and networks unique to RVL002 (FIGS. 3A-3E). The gene expression profiles of the brains of mice treated with vorinostat is largely due to its highly brain-permeable metabolite and this profile is closely mimicked by RVL002 while RVL002 increased bioavailability and biodistribution is reflected in a proportionally more extensive number of genes whose expression levels are altered. The top 4 pathways are associated with MeCP2 despite using wild-type C57B/L6 mice (Table 2), and several other pathways associated with MeCP2 are identified, albeit with lower statistical significance (Table 3). Overall, this indicates a MeCP2-pathway-mediated effect of RVL002.

TABLE 1
Pharmacokinetic studies in C57BL/6J mice dosed intraperitoneally with
Vorinostat (50 mg/kg) or RVL002 (50 mg/kg), over the course of 7 days.

| | | Vorinostat | | RVL002 | | RVL002/ |
|---|---|---|---|---|---|---|
| PK | | 50 mg/ml | | 50 mg/ml | | vorinostat |
| values | Unit | Mean | SD | Mean | SD | Ratio |
| T_{1/2} | h | 1.3 | 0.6 | 3.3 | 0.4 | 2.6 |
| Tₘₐₓ | h | 0.3 | 0.1 | 0.3 | 0.0 | 0.8 |
| Cₘₐₓ | ng/ml | 713 | 480 | 12280 | 1814 | 17 |
| AUCₗₐₛₜ | h*ng/ml | 632 | 452 | 11779 | 1890 | 19 |
| AUC_{inf} | h*ng/ml | 640 | 461 | 11649 | 2218 | 18 |

TABLE 2
Top 10 Reactome pathways identified by predicting drugs
that mimic RVL002 or vorinostat, taking calculated interacting
gene sets, and identifying genes unique to RVL002.

| Pathway name | # Entities | pValue | # Reactions |
|---|---|---|---|
| Transcriptional | 26 | 2.33E−04 | 47 |
| regulation by RUNX3 | | | |
| KEAP1-NFE2L2 pathway | 29 | 3.22E−04 | 37 |
| RUNX3 Regulates Immune | 6 | 5.97E−04 | 5 |
| Response and Cell Migration | | | |
| MECP2 regulates | 6 | 5.97E−04 | 6 |
| transcription factors | | | |
| Deubiquitination | 48 | 6.57E−04 | 44 |
| Unwinding of DNA | 6 | 0.00150868 | 3 |
| Regulation of HMOX1 | 4 | 0.00337694 | 4 |
| expression and activity | | | |
| Displacement of DNA | 5 | 0.0036812 | 4 |
| glycosylase by APEX1 | | | |
| G2/M Checkpoints | 27 | 0.0042105 | 12 |
| SCF-beta-TrCP mediated | 13 | 0.00445912 | 3 |
| degradation of Emi1 | | | |

TABLE 3
Reactome pathways identified to MeCP2 by identifying genes responding to RVL002.

| | #Entities | pValue | #Reactions |
|---|---|---|---|
| MeCP2-related pathway names from muscle tissues | | | |
| Transcriptional Regulation by MECP2 | 20 | 3.70E−02 | 32 |
| MECP2 regulates transcription factors | 4 | 4.11E−02 | 4 |
| Regulation of MECP2 expression and activity | 9 | 6.74E−02 | 8 |
| Loss of MECP2 binding ability to the NCoR/SMRT complex | 3 | 8.47E−02 | 1 |
| Loss of function of MECP2 in Rett syndrome | 4 | 1.52E−01 | 2 |
| MECP2 regulates neuronal receptors and channels | 6 | 2.30E−01 | 10 |
| Loss of MECP2 binding ability to 5mC-DNA | 1 | 4.73E−01 | 1 |
| MECP2 regulates transcription of neuronal ligands | 2 | 4.96E−01 | 4 |
| MeCP2-related pathway names from brain tissues | | | |
| MECP2 regulates transcription factors | 4 | 2.03E−05 | 4 |
| Transcriptional Regulation by MECP2 | 6 | 4.90E−03 | 13 |
| MECP2 regulates transcription of neuronal ligands | 1 | 1.82E−01 | 2 |
| MECP2 regulates neuronal receptors and channels | 1 | 3.90E−01 | 2 |
| Regulation of MECP2 expression and activity | 1 | 4.52E−01 | 3 |

### Example 6-Demonstration of Succinanilic Acid (RVL002) for Treating Rett Syndrome in Animal Models

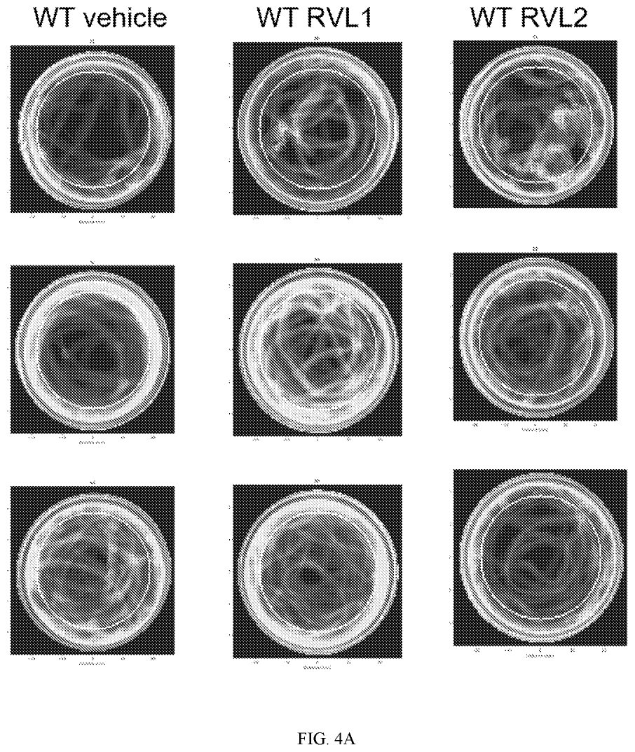

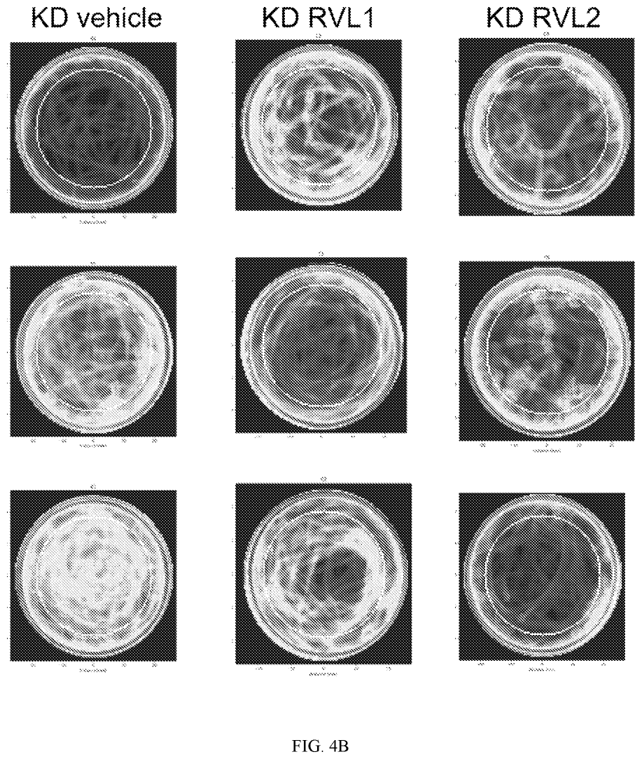

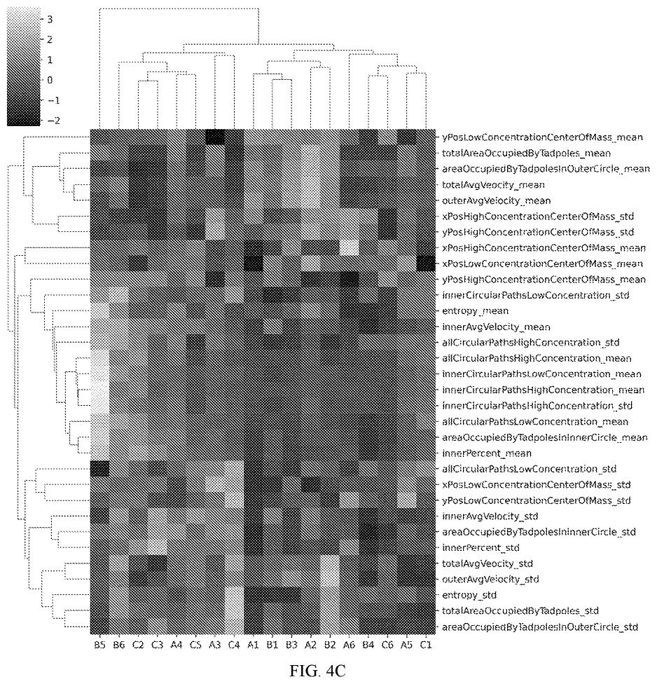

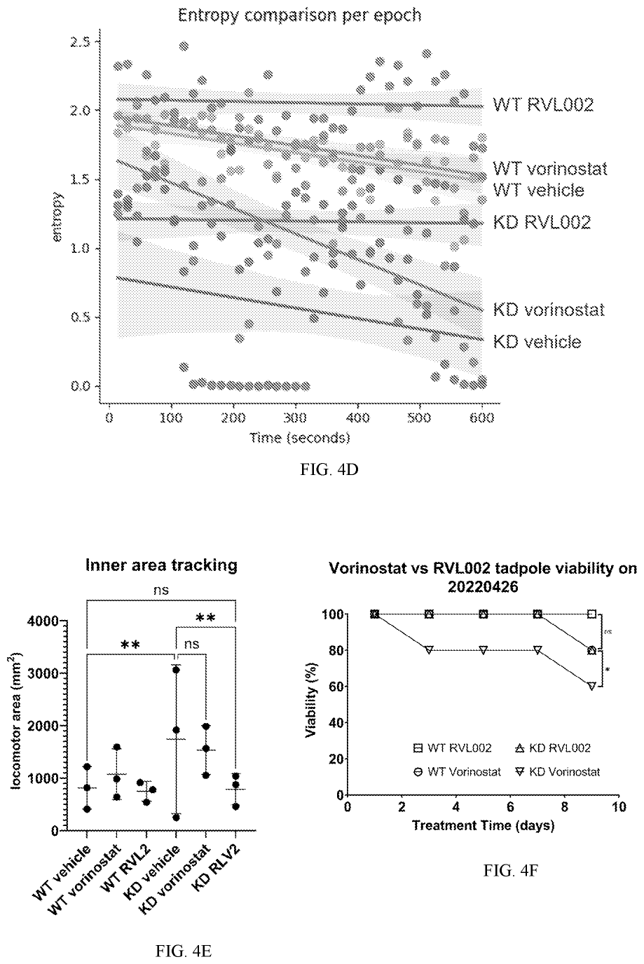

Using our tadpole tracking system, we evaluated the effect of vorinostat and RVL002 on WT and RTT tadpoles swimming behavior (FIGS. 4A-4F). WT tadpoles spend most of the time swimming along the outside of the dish and only occasionally cross through the center of the dish, while RTT tadpoles swim more erratically and tend to dart through the center of the dish and stop more frequently (FIG. 4A-4B, n=5 per dish). These abnormal swimming patterns could be related to motor irregularities and seizures seen in human RTT patients. RTT tadpoles treated with RVL002 adopt more circular motion and stop less frequently compared with vorinostat where patterns suggestive of darting and seizure are still observed. The entropy captures deviations in swimming behavior, including distance and area explored. The entropy plot suggests RVL002 rescue the behavior of RTT tadpoles (FIG. 4C-4D), each point represents the swimming behavior of 5 tadpoles in a dish during a 15-second window). RTT KD tadpole groups dosed with 50 μM RVL002 showed a 78% reduction in inner dish circular swimming compared to vehicle dosed KDs and 5 μM vorinostat led to a 42% reduction in average inner dish swimming compared to vehicle dosed KDs suggesting they are adopting a more typical behavior (FIG. 4E). We also observed that RVL002 (100 uM) is well tolerated and improves the viability of RTT tadpoles compared with vorinostat (50 uM) (FIG. 4F, n=10 per condition). We compared the gene expression signatures of vorinostat- and RVL002-treated tadpole RTT model animals and wild type controls. In wild type animals, 100% of genes with significant expression changes (fold change >2, p<0.05) due to vorinostat treatment were accounted for by RVL002 treatment. In RTT animals, RVL002 replicated 92.5% of gene expression changes. Therefore, RVL002, which lacks HDACi activity, can be used to successfully replicate the significant therapeutic benefits of vorinostat for RTT but without adverse effects associated with HDACi drugs.

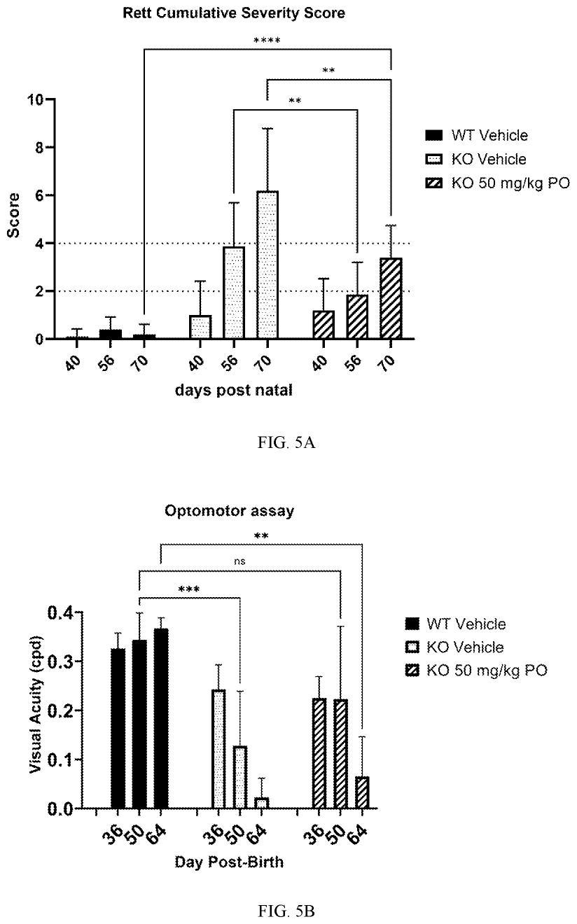

Mice (MeCP2-/y or WT littermates) were treated with 50 mg/kg P.O. RVL002 daily following the onset of severe symptoms as determined by the Rett cumulative severity scoring system (Guy et al., 2001, 2007). RVL002 was able to significantly reduce the severity of the Rett syndrome phenotypes compared to vehicle controls (FIGS. 5A-5B).

### Example 7-Treating Rett Syndrome and Other Disorders

Protein found to be direct targets of vorinostat using proteomic and serving as the basis for RVL002 and its analogs. Using proteomic methods, sixteen proteins, other than HDACs, were found to be directly impacted by the presence of vorinostat administered to Xenopus tadpoles (Table 4) (Novak et al., 2022). Curated databases and peer-reviewed literatures are useful to make a preliminary assessment of those hits, such as the OMIM knowledge database of gene and disease phenotype association (Amberger et al., 2019), Reactome knowledgebase, knowledge graph and analytical tool of molecular pathway interactions (Gillespie et al., 2022), String protein-protein interaction knowledgebase and visualization tool (von Mering et al., 2005), and PathwayCommon, an author-curated molecular interaction and pathway visualization tool (Rodchenkov et al., 2020).

Eight of those sixteen are known to be involved in or be regulated by acetylation, which was proposed to explain the normalization of the imbalance in tubulin acetylation observed across tissues in both the tadpoles mosaic for MeCP2 and the MeCP2-null mouse model for Rett syndrome. These include Protein diaphanous homolog 1 (DIAPHI), Prothymosin alpha (PTMA), the ATP synthase subunit alpha (ATP5F1A) and subunit gamma (ATP5F1C), the 40S ribosomal proteins S8 (RPS8) and S6 (RPS6), Histone H3.1 (H3C2), Cytoskeleton-associated protein 4 (CKAP4), Queuosine salvage protein (C9orf64), Acyl-CoA synthetase short-chain family member 3 (ACSS3), and the Transformation/transcription domain-associated protein (TRAPP). Additionally, many proteins function as part of ion transport and sensing, which is a group of targets that is often the subject of medicinal chemistry drug development programs in neurological disorders. Many of those have been associated with phenotypes seen in mitochondrial diseases, myopathies and neurodevelopmental disorders, such as such as seizures, hearing loss, intellectual disabilities, and autism. These include Trypsin-2 (PRSS2), ATP5F1A, ATP5F1C, Sodium-dependent multivitamin transporter (SLC5A6) and Extracellular calcium-sensing receptor (CASR), that last one being a G-protein coupled receptors, one of the most favorite class of protein to drug in the pharmaceutical industry. Several protein are natural candidates for degradation pathways using molecular glues or proximity ligands, such as PTMA, CASR, RPS6, RPS8, H3C2 and TRAPP. Lastly, lipid metabolism is also well represented by ATP5F1A, ATP5F1C, Phospholipid transfer protein (PLTP), RPS8, CKAP4, ACSS3, and SLC5A6. This information partly served as the motivation for developing and testing RVL002 and its analogs in the context of Rett Syndrome and other neurodevelopmental disorders.

ACSS3. Acyl-CoA synthetase short-chain family member 3 (ACSS3) performs the initial step in the short chain fatty acid metabolism, by catalyzing the formation of a thioester between short chain fatty acid and CoA (Watkins et al., 2007). While no disease has been clinically associated with ACSS3, or Acyl-CoA synthetases family members, it was recently shown to causes propionate to accumulate, leading to lipid accumulation, lower energy metabolism and systemic metabolic impairment, including lower oxygen consumption and homeostasis, in rat model of ACSS3 deficiency (Jia et al., 2022).

Several other proteins are involved in the complex short chain fatty acid metabolism with activities located both inside and on the periphery of the mitochondria (Watkins et al., 2007). Relevant to this example are proteins that have both demonstrated direct connections to ACSS3 and they have been associated with rare pediatric disorders bearing Rett syndrome-like phenotypes.

One example includes the Short-chain specific acyl-CoA dehydrogenase (ACADS) which is associated with Short-chain acyl-CoA dehydrogenase deficiency (OMIM: 201470). It is characterized by early neurologic impairment, myopathy, scoliosis, hypotonia, developmental delay, seizures. Changes in metabolism can be detected by measuring aciduria and accumulation of acyl and butyryl carnitine in the patients.

A second example includes Acetyl-CoA acetyltransferase (ACAT1), which is associated with alpha-methylacetoacetic aciduria (OMIM: 203750). It is characterized by normal development until 6 months old followed by mental retardation and ketoacidosis attack which manifest by severe intestinal disfunction, vomiting, dehydration, shortness of breath.

A third example includes Mitochondrial carnitine/acylcarnitine carrier protein (CACTD), which is associated with carnitine-acylcarnitine translocase deficiency (OMIM: 212138). It is characterized by early onset of neurologic abnormalities, myopathy, liver disfunction, seizures, apnea, bradycardia and a infantile high mortality rate and the most severe presentations. Changes in metabolism can be detected by measuring hypoglycemia, hyperammonemia, elevated creatine kinase, aciduria, and an abnormal carnitine profile in patients.

A fourth example includes Hydroxyacyl-coenzyme A dehydrogenase (HDAH), which is associated with 3-alpha-hydroxyacyl-CoA dehydrogenase deficiency (OMIM: 231530). It presents with similar characteristics of the previous three examples, such as myopathy, liver damage, seizures, neurological disorders, while measuring hypoglycemia, aciduria, along with decreased in the enzyme catalytic can serve as diagnosis.

A fifth example is Mitchell syndrome (OMIM: 618960), a progressive disorder characterized by demyelination, peripheral europathy, seizures, ataxia, respiratory failure and hearing loss. It is caused by peroxisomal acyl-CoA oxidase (ACOX1) deficiency, and enzyme involved in propionate metabolism pathway along with ACSS3 and ACADS.

Lipid metabolism and mitochondrial activity have been shown to be relevant to Rett syndrome and other neurodevelopmental disorders, including myopathies and mitochondrial diseases such as Leigh Syndromes, imbalance in fatty acid metabolism were recently shown in Fragile X syndrome (OMIM: 300624), which is caused by loss of function in Fragile X messenger ribonucleoprotein 1 (FMR1) which, like MeCP2, is not directly involved in fatty acid metabolism but as broad ranging impact (Abolghasemi et al., 2022). Carnitine metabolism was recently shown to be imbalanced in a MeCP2 heterozygous mouse model for Rett syndrome, and was proposed to lead to metabolic failure and early mortality in that model (Mucerino eta, 2017). Earlier work had shown that L-cairntine supplementation had modest but broad ranging benefit in the MeCP2-null male mouse model when administered from post-natal day 1 (P1) (Schaevitz etman, 2012).

The direct drug-target interaction between vorinostat and ACSS3 identified in animal models by Novak etaM (2022), and the intersection of genes and pathways modulated by RVL002, demonstrate that modulating short-chain fatty acid metabolism via ACSS3 can meaningfully rescue Rett syndrome phenotypes.

TABLE 4
Protein found to be direct targets of vorinostat using proteomic and serving as the basis for RVL002 and its analogs (Novak et al. 2022).

| Target | Description | Pathway |
|---|---|---|
| DIAPH1 | Protein diaphanous homolog 1 | Acetylation; Actin-binding; Alternative splicing; Cell membrane; |
| | | Cytoskeleton; Deafness; Epilepsy; Hearing |
| PTMA | Prothymosin alpha | Acetylation; Alternative splicing; Ubl conjugation |
| PRSS2 | Trypsin-2 | Calcium; Disulfide bond; Hydrolase; Metal-binding; Serine protease; |
| | | Zymogen |
| ATP5F1A | ATP synthase subunit alpha | Acetylation; Alternative splicing; ATP synthesis; Glycoprotein; Ion |
| | | transport; Methylation; Mitochondrion; Primary mitochondrial disease |
| ATP5F1C | ATP synthase subunit gamma | Acetylation; Alternative splicing; ATP synthesis; Ion transport; |
| | | Mitochondrion |
| CASR | Extracellular calcium-sensing receptor | Alternative splicing; Calcium; Disease variant; Disulfide bond; Epilepsy; G- |
| | | protein coupled receptor; Glycoprotein; Metal-binding; Transmembrane |
| | | helix; Ubl conjugation |
| PLTP | Phospholipid transfer protein | Alternative splicing; Disulfide bond; Glycoprotein; Lipid transport; |
| | | Nucleus; Secreted |
| RPS8 | 40S ribosomal protein S8 | Acetylation; Isopeptide bond; Lipoprotein; Membrane; Myristate; |
| | | Ribosomal protein; Ubl conjugation |
| RPS6 | 40S ribosomal protein S6 | Acetylation; ADP-ribosylation; Hydroxylation; Isopeptide bond; |
| | | Ribosomal protein; Ubl conjugation |
| H3C2 | Histone H3.1 | Acetylation; ADP-ribosylation; Citrullination; Disease variant; DNA- |
| | | binding; Hydroxylation; Methylation; Nucleosome; Nucleus; Ubl |
| | | conjugation |
| CKAP4 | Cytoskeleton-associated protein 4 | Acetylation; Cell membrane; Cytoskeleton; Endoplasmic reticulum; |
| | | Lipoprotein; Palmitate; Signal-anchor; Transmembrane helix |
| C9orf64 | Queuosine salvage protein | Acetylation |
| NOC4L | Nucleolar complex protein 4 homolog | Nucleus; Transmembrane helix |
| ACSS3 | Acyl-CoA synthetase short-chain family member 3 | Acetylation; Alternative splicing; ATP-binding; Ligase; Lipid metabolism; |
| | | Mitochondrion |
| SLC5A6 | Sodium-dependent multivitamin transporter | Biotin; Cell membrane; Glycoprotein; Ion transport; Membrane; |
| | | Neurodegeneration; Sodium transport; Symport; Transmembrane helix |
| TRRAP | Transformation/transcription domain-associated protein | Acetylation; Activator; Alternative splicing; Autism spectrum disorder; |
| | | Chromatin regulator; Deafness; Intellectual disability; Isopeptide bond; |
| | | Transcription regulation; Ubl conjugation |

Several proteins are associated with diseases presenting phenotypes similar to phenotypes observed with Rett syndrome. These diseases can include hypotonia with psychomotor retardation, polyendocrine syndrome with or without dysplasia, barter syndrome, oxidative phosphorylation deficiency, deafness with or without thrombocytopenia, developmental delay with or without dysmorphic facies and autism, epilepsy idiopathic, familial hypocalciuric hypercalcemia, hypocalcemia, hypoparathyroidism with sensorineural deafness and renal dysplasia syndrome, melanoma susceptibility, microcephaly syndrome, mitochondrial complex V ATP synthase deficiency, multiple endocrine neoplasia, pediatric peripheral motor neuropathy, polyendocrine syndrome with or without dysplasia, schizophrenia, seizures, neonatal hyperparathyroidism, sodium-dependent multivitamin transporter.

### Example 8-Demonstration of Succinanilic Acid (Rvl002) Acting Through Acss3 for Treating Rett Syndrome in Animal Models

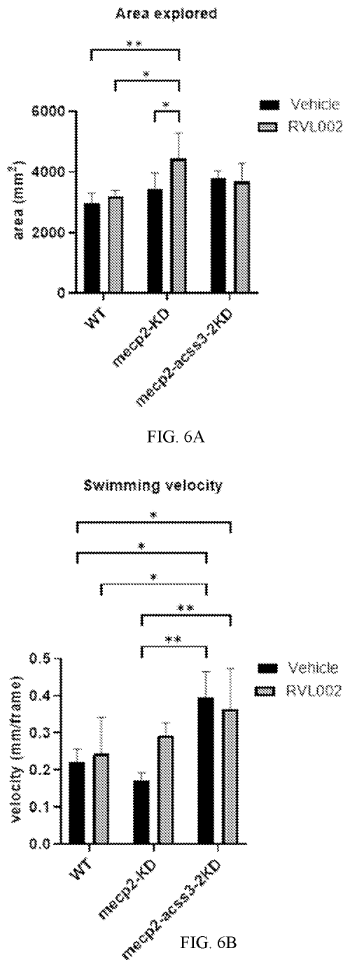

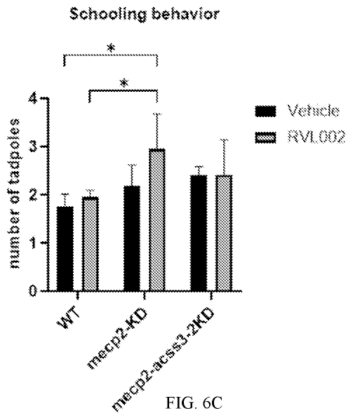

Using our tadpole tracking system, we evaluated the effect of RVL002 on WT, RTT and tadpoles deficient for both MeCP2 and ACSS3 (FIGS. 6A-6C). Wild-type (WT), MeCP2 knockdown (mecp2-KD) and double knockdown of MeCP2 and ACSS3 (mecp2-acss3-2KD) tadpoles, were compared in the presence of absence of 50 μM RVL002 added to the swimming media every other day starting at 6 dpf and recorded for 20 minutes at 15 dpf Mecp2-acss3-2KD tadpoles did not respond to RVL002 across exploratory behavior (FIG. 6A), swimming velocity (FIG. 6B) and the number of tadpoles in a school (FIG. 6C). This demonstrates that in an animal model for Rett syndrome, ACSS3 is required to respond to RVL002.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

Abel, T. and Zukin, R. S. (2008) “Epigenetic targets of HDAC inhibition in neurodegenerative and psychiatric disorders.,” Current Opinion in Pharmacology, 8(1), pp. 57-64. doi: 10.1016/j.coph.2007.12.002.
Abolghasemi, A., Carullo, M. P., Aguilera, E. C., Laroui, A., Plantefeve, R., Rojas, D., Benachenhou, S., Ramirez, M. V., Proteau-Lemieux, M., Lepage, J.-F., Corbin, F., Plourde, M., Farez, M., Cogram, P. and Qaku, A. (2022) “Alteration of fatty acid profile in fragile X syndrome.,” International Journal of Molecular Sciences, 23(18). doi: 10.3390/ijms231810815.
Amberger, J. S., Bocchini, C. A., Scott, A. F. and Hamosh, A. (2019) “OMIM.org: leveraging knowledge across phenotype-gene relationships.,” Nucleic Acids Research, 47(D1), pp. D1038-D1043. doi: 10.1093/nar/gky1151.
Amir, R. E., Van den Veyver, I. B., Wan, M., Tran, C. Q., Francke, U. and Zoghbi, H. Y. (1999) “Rett syndrome is caused by mutations in X-linked MECP2, encoding methyl-CpG-binding protein 2.,” Nature Genetics, 23(2), pp. 185-188. doi: 10.1038/13810.
Anderson, A., Wong, K., Jacoby, P., Downs, J. and Leonard, H. (2014) “Twenty years of surveillance in Rett syndrome: what does this tell us?,” Orphanet Journal of Rare Diseases, 9, p. 87. doi: 10.1186/1750-1172-9-87.
Anitha, A., Poovathinal, S. A., Viswambharan, V., Thanseem, I., Iype, M., Anoop, U., Sumitha, P. S., Parakkal, R. and Vasu, M. M. (2022) “MECP2 Mutations in the Rett Syndrome Patients from South India.,” Neurology India, 70(1), pp. 249-253. doi: 10.4103/0028-3886.338714.
Basu, T., O'Riordan, K. J., Schoenike, B. A., Khan, N. N., Wallace, E. P., Rodriguez, G., Maganti, R. K. and Roopra, A. (2019) “Histone deacetylase inhibitors restore normal hippocampal synaptic plasticity and seizure threshold in a mouse model of Tuberous Sclerosis Complex.,” Scientific Reports, 9(1), p. 5266. doi: 10.1038/s41598-019-41744-7.
Benito, E., Urbanke, H., Ramachandran, B., Barth, J., Halder, R., Awasthi, A., Jain, G., Capece, V., Burkhardt, S., Navarro-Sala, M., Nagarajan, S., Schütz, A.-L., Johnsen, S. A., Bonn, S., Lührmann, R., Dean, C. and Fischer, A. (2015) “HDAC inhibitor-dependent transcriptome and memory reinstatement in cognitive decline models.,” The Journal of Clinical Investigation, 125(9), pp. 3572-3584. doi: 10.1172/JCI79942.
Borloz, E., Villard, L. and Roux, J.-C. (2021) “Rett syndrome: think outside the (skull) box.,” Faculty Reviews, 10, p. 59. doi: 10.12703/r/10-59.
Braunschweig, D., Simcox, T., Samaco, R. C. and LaSalle, J. M. (2004) “X-Chromosome inactivation ratios affect wild-type MeCP2 expression within mosaic Rett syndrome and Mecp2-/+ mouse brain.,” Human Molecular Genetics, 13(12), pp. 1275-1286. doi: 10.1093/hmg/ddhl42.
Buchanan, C. B., Stallworth, J. L., Scott, A. E., Glaze, D. G., Lane, J. B., Skinner, S. A., Tierney, A. E., Percy, A. K., Neul, J. L. and Kaufmann, W. E. (2019) “Behavioral profiles in Rett syndrome: Data from the natural history study.,” Brain & Development, 41(2), pp. 123-134. doi: 10.1016/j.braindev.2018.08.008.
Cao, F., Zwinderman, M. R. H. and Dekker, F. J. (2018) “The process and strategy for developing selective histone deacetylase 3 inhibitors.,” Molecules (Basel, Switzerland), 23(3). doi: 10.3390/molecules23030551.
Chahrour, M., Jung, S. Y., Shaw, C., Zhou, X., Wong, S. T. C., Qin, J. and Zoghbi, H. Y. (2008) “MeCP2, a key contributor to neurological disease, activates and represses transcription.,” Science, 320(5880), pp. 1224-1229. doi: 10.1126/science.1153252.
Citraro, R., Leo, A., Santoro, M., D'agostino, G., Constanti, A. and Russo, E. (2017) “Role of histone deacetylases (hdacs) in epilepsy and epileptogenesis.,” Current Pharmaceutical Design, 23(37), pp. 5546-5562. doi: 10.2174/1381612823666171024130001.
Colvin, L., Fyfe, S., Leonard, S., Schiavello, T., Ellaway, C., De Klerk, N., Christodoulou, J., Msall, M. and Leonard, H. (2003) “Describing the phenotype in Rett syndrome using a population database.,” Archives of Disease in Childhood, 88(1), pp. 38-43. doi: 10.1136/adc.88.1.38.
Cuddapah, V. A., Pillai, R. B., Shekar, K. V., Lane, J. B., Motil, K. J., Skinner, S. A., Tarquinio, D. C., Glaze, D. G., McGwin, G., Kaufmann, W. E., Percy, A. K., Neul, J. L. and Olsen, M. L. (2014) “Methyl-CpG-binding protein 2 (MECP2) mutation type is associated with disease severity in Rett syndrome.,” Journal of Medical Genetics, 51(3), pp. 152-158. doi: 10.1136/jmedgenet-2013-102113.
De Felice, C., Rossi, M., Leoncini, S., Chisci, G., Signorini, C., Lonetti, G., Vannuccini, L., Spina, D., Ginori, A., Iacona, I., Cortelazzo, A., Pecorelli, A., Valacchi, G., Ciccoli, L., Pizzorusso, T. and Hayek, J. (2014) “Inflammatory lung disease in Rett syndrome.,” Mediators of Inflammation, 2014, p. 560120. doi: 10.1155/2014/560120.
Ehrhart, F., Coort, S. L., Eijssen, L., Cirillo, E., Smeets, E. E., Bahram Sangani, N., Evelo, C. T. and Curfs, L. M. G. (2020) “Integrated analysis of human transcriptome data for Rett syndrome finds a network of involved genes.,” The World Journal of Biological Psychiatry, 21(10), pp. 712-725. doi: 10.1080/15622975.2019.1593501.
Ehrhart, F., Jacobsen, A., Rigau, M., Bosio, M., Kaliyaperumal, R., Laros, J. F. J., Willighagen, E. L., Valencia, A., Roos, M., Capella-Gutierrez, S., Curfs, L. M. G. and Evelo, C. T. (2021) “A catalogue of 863 Rett-syndrome-causing MECP2 mutations and lessons learned from data integration.,” Scientific data, 8(1), p. 10. doi: 10.1038/s41597-020-00794-7.
Ellaway, C., Peat, J., Leonard, H. and Christodoulou, J. (2001) “Sleep dysfunction in Rett syndrome: lack of age related decrease in sleep duration.,” Brain & Development, 23 Suppl 1, pp. S101-3. doi: 10.1016/SO387-7604(01)00356-4.
Fehr, S., Bebbington, A., Nassar, N., Downs, J., Ronen, G. M., DE Klerk, N. and Leonard, H. (2011) “Trends in the diagnosis of Rett syndrome in Australia.,” Pediatric Research, 70(3), pp. 313-319. doi: 10.1203/PDR.0b013e3182242461.
Fischer, A., Sananbenesi, F., Mungenast, A. and Tsai, L.-H. (2010) “Targeting the correct HDAC(s) to treat cognitive disorders.,” Trends in Pharmacological Sciences, 31(12), pp. 605-617. doi: 10.1016/j.tips.2010.09.003.
Frullanti, E., Papa, F. T., Grillo, E., Clarke, A., Ben-Zeev, B., Pineda, M., Bahi-Buisson, N., Bienvenu, T., Armstrong, J., Roche Martinez, A., Mari, F., Nissenkorn, A., Lo Rizzo, C., Veneselli, E., Russo, S., Vignoli, A., Pini, G., Djuric, M., Bisgaard, A.-M., Ravn, K., Bosnjak, V. M., Hayek, J., Khajuria, R., Montomoli, B., Cogliati, F., Pintaudi, M., Hadzsiev, K., Craiu, D., Voinova, V., Djukic, A., Villard, L. and Renieri, A. (2019) “Analysis of the phenotypes in the rett networked database.,” International journal of genomics, 2019, p. 6956934. doi: 10.1155/2019/6956934.
Fu, C., Armstrong, D., Marsh, E., Lieberman, D., Motil, K., Witt, R., Standridge, S., Lane, J., Dinkel, T., Jones, M., Hale, K., Suter, B., Glaze, D., Neul, J., Percy, A. and Benke, T. (2020) “Multisystem comorbidities in classic Rett syndrome: a scoping review.,” BMJ Paediatrics Open, 4(1), p. e000731. doi: 10.1136/bmjpo-2020-000731.
Fu, C., Armstrong, D., Marsh, E., Lieberman, D., Motil, K., Witt, R., Standridge, S., Nues, P., Lane, J., Dinkel, T., Coenraads, M., von Hehn, J., Jones, M., Hale, K., Suter, B., Glaze, D., Neul, J., Percy, A. and Benke, T. (2020) “Consensus guidelines on managing Rett syndrome across the lifespan.,” BMJ Paediatrics Open, 4(1), p. e000717. doi: 10.1136/bmjpo-2020-000717.
Gillespie, M., Jassal, B., Stephan, R., Milacic, M., Rothfels, K., Senff-Ribeiro, A., Griss, J., Sevilla, C., Matthews, L., Gong, C., Deng, C., Varusai, T., Ragueneau, E., Haider, Y., May, B., Shamovsky, V., Weiser, J., Brunson, T., Sanati, N., Beckman, L., Shao, X., Fabregat, A., Sidiropoulos, K., Murillo, J., Viteri, G., Cook, J., Shorser, S., Bader, G., Demir, E., Sander, C., Haw, R., Wu, G., Stein, L., Hermjakob, H. and D'Eustachio, P. (2022) “The reactome pathway knowledgebase 2022.,” Nucleic Acids Research, 50(D1), pp. D687-D692. doi: 10.1093/nar/gkab1028.
Guy, J., Gan, J., Selfridge, J., Cobb, S. and Bird, A. (2007) “Reversal of neurological defects in a mouse model of Rett syndrome.,” Science, 315(5815), pp. 1143-1147. doi: 10.1126/science.1138389.
Guy, J., Hendrich, B., Holmes, M., Martin, J. E. and Bird, A. (2001) “A mouse Mecp2-null mutation causes neurological symptoms that mimic Rett syndrome.,” Nature Genetics, 27(3), pp. 322-326. doi: 10.1038/85899.
Hagberg, B. (2002) “Clinical manifestations and stages of Rett syndrome.,” Mental retardation and developmental disabilities research reviews, 8(2), pp. 61-65. doi: 10.1002/mrdd.10020.
Hauke, J., Riessland, M., Lunke, S., Eyüpoglu, I. Y., Blumcke, I., El-Osta, A., Wirth, B. and Hahnen, E. (2009) “Survival motor neuron gene 2 silencing by DNA methylation correlates with spinal muscular atrophy disease severity and can be bypassed by histone deacetylase inhibition.,” Human Molecular Genetics, 18(2), pp. 304-317. doi: 10.1093/hmg/ddn357.
Hendrie, D., Bebbington, A., Bower, C. and Leonard, H. (2011) “Measuring use and cost of health sector and related care in a population of girls and young women with Rett syndrome,” Research in autism spectrum disorders, 5(2), pp. 901-909. doi: 10.1016/j.rasd.2010.10.004.
Henriksen, M. W., Breck, H., von Tetzchner, S., Paus, B. and Skjeldal, O. H. (2020) “Medical Issues in Adults with Rett Syndrome—A National Survey.,” Developmental neurorehabilitation, 23(2), pp. 106-112. doi: 10.1080/17518423.2019.1646341.
Ivy, A. S. and Standridge, S. M. (2021) “Rett syndrome: A timely review from recognition to current clinical approaches and clinical study updates.,” Seminars in pediatric neurology, 37, p. 100881. doi: 10.1016/j.spen.2021.100881.
Jia, Z., Chen, X., Chen, J., Zhang, L., Oprescu, S. N., Luo, N., Xiong, Y., Yue, F. and Kuang, S. (2022) “ACSS3 in brown fat drives propionate catabolism and its deficiency leads to autophagy and systemic metabolic dysfunction.,” Clinical and translational medicine, 12(2), p. e665. doi: 10.1002/ctm2.665.
Katz, D. M., Berger-Sweeney, J. E., Eubanks, J. H., Justice, M. J., Neul, J. L., Pozzo-Miller, L., Blue, M. E., Christian, D., Crawley, J. N., Giustetto, M., Guy, J., Howell, C. J., Kron, M., Nelson, S. B., Samaco, R. C., Schaevitz, L. R., St Hillaire-Clarke, C., Young, J. L., Zoghbi, H. Y. and Mamounas, L. A. (2012) “Preclinical research in Rett syndrome: setting the foundation for translational success.,” Disease Models & Mechanisms, 5(6), pp. 733-745. doi: 10.1242/dmm.011007.
Katz, D. M., Bird, A., Coenraads, M., Gray, S. J., Menon, D. U., Philpot, B. D. and Tarquinio, D. C. (2016) “Rett syndrome: crossing the threshold to clinical translation.,” Trends in Neurosciences, 39(2), pp. 100-113. doi: 10.1016/j.tins.2015.12.008.
Keenan, A. B., Jenkins, S. L., Jagodnik, K. M., Koplev, S., He, E., Torre, D., Wang, Z., Dohlman, A. B., Silverstein, M. C., Lachmann, A., Kuleshov, M. V., Ma'ayan, A., Stathias, V., Terryn, R., Cooper, D., Forlin, M., Koleti, A., Vidovic, D., Chung, C., Schurer, S. C., Vasiliauskas, J., Pilarczyk, M., Shamsaei, B., Fazel, M., Ren, Y., Niu, W., Clark, N. A., White, S., Mahi, N., Zhang, L., Kouril, M., Reichard, J. F., Sivaganesan, S., Medvedovic, M., Meller, J., Koch, R. J., Birtwistle, M. R., Iyengar, R., Sobie, E. A., Azeloglu, E. U., Kaye, J., Osterloh, J., Haston, K., Kalra, J., Finkbiener, S., Li, J., Milani, P., Adam, M., Escalante-Chong, R., Sachs, K., Lenail, A., Ramamoorthy, D., Fraenkel, E., Daigle, G., Hussain, U., Coye, A., Rothstein, J., Sareen, D., Ornelas, L., Banuelos, M., Mandefro, B., Ho, R., Svendsen, C. N., Lim, R. G., Stocksdale, J., Casale, M. S., Thompson, T. G., Wu, J., Thompson, L. M., Dardov, V., Venkatraman, V., Matlock, A., Van Eyk, J. E., Jaffe, J. D., Papanastasiou, M., Subramanian, A., Golub, T. R., Erickson, S. D., Fallahi-Sichani, M., Hafner, M., Gray, N. S., Lin, J.-R., Mills, C. E., Muhlich, J. L., Niepel, M., Shamu, C. E., Williams, E. H., Wrobel, D., Sorger, P. K., Heiser, L. M., Gray, J. W., Korkola, J. E., Mills, G. B., LaBarge, M., Feiler, H. S., Dane, M. A., Bucher, E., Nederlof, M. and Pillai, A. (2018) “The Library of Integrated Network-Based Cellular Signatures NIH Program: System-Level Cataloging of Human Cells Response to Perturbations.,” Cell Systems, 6(1), pp. 13-24. doi: 10.1016/j.cels.2017.11.001.
Kerr, A. M., Nomura, Y., Armstrong, D., Anvret, M., Belichenko, P. V., Budden, S., Cass, H., Christodoulou, J., Clarke, A., Ellaway, C., d'Esposito, M., Francke, U., Hulten, M., Julu, P., Leonard, H., Naidu, S., Schanen, C., Webb, T., Engerstrom, I. W., Yamashita, Y. and Segawa, M. (2001) “Guidelines for reporting clinical features in cases with MECP2 mutations.,” Brain & Development, 23(4), pp. 208-211. doi: 10.1016/s0387-7604(01)00193-0.
Kerr, A. M. and Prescott, R. J. (2005) “Predictive value of the early clinical signs in Rett disorder.,” Brain & Development, 27 Suppl 1, pp. S20-S24. doi: 10.1016/j.braindev.2004.10.007.
Krishnaraj, R., Haase, F., Coorey, B., Luca, E. J., Wong, I., Boyling, A., Ellaway, C., Christodoulou, J. and Gold, W. A. (2019) “Genome-wide transcriptomic and proteomic studies of Rett syndrome mouse models identify common signaling pathways and cellular functions as potential therapeutic targets.,” Human Mutation, 40(12), pp. 2184-2196. doi: 10.1002/humu.23887.
Kyle, S. M., Vashi, N. and Justice, M. J. (2018) “Rett syndrome: a neurological disorder with metabolic components.,” Open biology, 8(2). doi: 10.1098/rsob.170216.
Landucci, E., Brindisi, M., Bianciardi, L., Catania, L. M., Daga, S., Croci, S., Frullanti, E., Fallerini, C., Butini, S., Brogi, S., Furini, S., Melani, R., Molinaro, A., Lorenzetti, F. C., Imperatore, V., Amabile, S., Mariani, J., Mari, F., Ariani, F., Pizzorusso, T., Pinto, A. M., Vaccarino, F. M., Renieri, A., Campiani, G. and Meloni, I. (2018) “iPSC-derived neurons profiling reveals GABAergic circuit disruption and acetylated α-tubulin defect which improves after iHDAC6 treatment in Rett syndrome.,” Experimental Cell Research, 368(2), pp. 225-235. doi: 10.1016/j.yexcr.2018.05.001.
Li, S., Lu, X., Shao, Q., Chen, Z., Huang, Q., Jiao, Z., Huang, X., Yue, M., Peng, J., Zhou, X., Chao, D., Zhao, H., Ji, J., Ji, Y. and Ji, Q. (2019) “Early histone deacetylase inhibition mitigates ischemia/reperfusion brain injury by reducing microglia activation and modulating their phenotype.,” Frontiers in neurology, 10, p. 893. doi: 10.3389/fneur.2019.00893.
Lyst, M. J., Ekiert, R., Ebert, D. H., Merusi, C., Nowak, J., Selfridge, J., Guy, J., Kastan, N. R., Robinson, N. D., de Lima Alves, F., Rappsilber, J., Greenberg, M. E. and Bird, A. (2013) “Rett syndrome mutations abolish the interaction of MeCP2 with the NCoR/SMRT co-repressor.,” Nature Neuroscience, 16(7), pp. 898-902. doi: 10.1038/nn.3434.
Mahgoub, M., Adachi, M., Suzuki, K., Liu, X., Kavalali, E. T., Chahrour, M. H. and Monteggia, L. M. (2016) “MeCP2 and histone deacetylases 1 and 2 in dorsal striatum collectively suppress repetitive behaviors.,” Nature Neuroscience, 19(11), pp. 1506-1512. doi: 10.1038/nn.4395.
von Mering, C., Jensen, L. J., Snel, B., Hooper, S. D., Krupp, M., Foglierini, M., Jouffre, N., Huynen, M. A. and Bork, P. (2005) “STRING: known and predicted protein-protein associations, integrated and transferred across organisms.,” Nucleic Acids Research, 33(Database issue), pp. D433-7. doi: 10.1093/nar/gki005.
Mucerino, S., Di Salle, A., Alessio, N., Margarucci, S., Nicolai, R., Melone, M. A. B., Galderisi, U. and Peluso, G. (2017) “Alterations in the carnitine cycle in a mouse model of Rett syndrome.,” Scientific Reports, 7, p. 41824. doi: 10.1038/srep41824.
Nan, X., Ng, H. H., Johnson, C. A., Laherty, C. D., Turner, B. M., Eisenman, R. N. and Bird, A. (1998) “Transcriptional repression by the methyl-CpG-binding protein MeCP2 involves a histone deacetylase complex.,” Nature, 393(6683), pp. 386-389. doi: 10.1038/30764.
Napoletani, G., Vigli, D., Cosentino, L., Grieco, M., Talamo, M. C., Lacivita, E., Leopoldo, M., Laviola, G., Fuso, A., d'Erme, M. and De Filippis, B. (2021) “Stimulation of the serotonin receptor 7 restores brain histone H3 acetylation and mecp2 corepressor protein levels in a female mouse model of rett syndrome.,” Journal of Neuropathology and Experimental Neurology, 80(3), pp. 265-273. doi: 10.1093/jnen/n1aa158.
Nielsen, J. B., Henriksen, K. F., Hansen, C., Silahtaroglu, A., Schwartz, M. and Tommerup, N. (2001) “MECP2 mutations in Danish patients with Rett syndrome: high frequency of mutations but no consistent correlations with clinical severity or with the X chromosome inactivation pattern.,” European Journal of Human Genetics, 9(3), pp. 178-184. doi: 10.1038/sj.ejhg.5200600.
Novak, R., Lin, T., Kaushal, S., Sperry, M. M., Vigneault, F., Gardner, E., Loomba, S., Shcherbina, K., Keshari, V., Dinis, A., Vasan, A., Chandrasekhar, V., Takeda, T., Turner, J. R., Levin, M. and Ingber, D. E. (2022) “Target-agnostic discovery of Rett Syndrome therapeutics by coupling computational network analysis and CRISPR-enabled in vivo disease modeling,” BioRxiv. doi: 10.1101/2022.03.20.485056.
Pecorelli, A., Cervellati, C., Cordone, V., Hayek, J. and Valacchi, G. (2020) “Compromised immune/inflammatory responses in Rett syndrome.,” Free Radical Biology & Medicine, 152, pp. 100-106. doi: 10.1016/j.freeradbiomed.2020.02.023.
Pecorelli, A., Cervellati, F., Belmonte, G., Montagner, G., Waldon, P., Hayek, J., Gambari, R. and Valacchi, G. (2016) “Cytokines profile and peripheral blood mononuclear cells morphology in Rett and autistic patients.,” Cytokine, 77, pp. 180-188. doi: 10.1016/j.cyto.2015.10.002.
Petrozziello, T., Dios, A. M., Mueller, K. A., Vaine, C. A., Hendriks, W. T., Glajch, K. E., Mills, A. N., Mangkalaphiban, K., Penney, E. B., Ito, N., Fernandez-Cerado, C., Legarda, G. P. A., Velasco-Andrada, M. S., Acuna, P. J., Ang, M. A., Munoz, E. L., Diesta, C. C. E., Macalintal-Canlas, R., Acuna, G., Sharma, N., Ozelius, L. J., Bragg, D. C. and Sadri-Vakili, G. (2020) “SVA insertion in X-linked Dystonia Parkinsonism alters histone H3 acetylation associated with TAF1 gene.,” Plos One, 15(12), p. e0243655. doi: 10.1371/journal.pone.0243655.
Renthal, W., Boxer, L. D., Hrvatin, S., Li, E., Silberfeld, A., Nagy, M. A., Griffith, E. C., Vierbuchen, T. and Greenberg, M. E. (2018) “Characterization of human mosaic Rett syndrome brain tissue by single-nucleus RNA sequencing.,” Nature Neuroscience, 21(12), pp. 1670-1679. doi: 10.1038/s41593-018-0270-6.
Rett, A. (1966) “[On a unusual brain atrophy syndrome in hyperammonemia in childhood].,” Wiener medizinische Wochenschrift (1946), 116(37), pp. 723-726.
Ribeiro, M. C. and MacDonald, J. L. (2020) “Sex differences in Mecp2-mutant Rett syndrome model mice and the impact of cellular mosaicism in phenotype development.,” Brain Research, 1729, p. 146644. doi: 10.1016/j.brainres.2019.146644.
Rietveld, L., Stuss, D. P., McPhee, D. and Delaney, K. R. (2015) “Genotype-specific effects of Mecp2 loss-of-function on morphology of Layer V pyramidal neurons in heterozygous female Rett syndrome model mice.,” Frontiers in Cellular Neuroscience, 9, p. 145. doi: 10.3389/fncel.2015.00145.
Rodchenkov, I., Babur, O., Luna, A., Aksoy, B. A., Wong, J. V., Fong, D., Franz, M., Siper, M. C., Cheung, M., Wrana, M., Mistry, H., Mosier, L., Dlin, J., Wen, Q., O'Callaghan, C., Li, W., Elder, G., Smith, P. T., Dallago, C., Cerami, E., Gross, B., Dogrusoz, U., Demir, E., Bader, G. D. and Sander, C. (2020) “Pathway Commons 2019 Update: integration, analysis and exploration of pathway data.,” Nucleic Acids Research, 48(D1), pp. D489-D497. doi: 10.1093/nar/gkz946.
Schaevitz, L. R., Nicolai, R., Lopez, C. M., D'Iddio, S., Iannoni, E. and Berger-Sweeney, J. E. (2012) “Acetyl-L-carnitine improves behavior and dendritic morphology in a mouse model of Rett syndrome.,” Plos One, 7(12), p. e51586. doi: 10.1371/journal.pone.0051586.
Shahbazian, M., Young, J., Yuva-Paylor, L., Spencer, C., Antalffy, B., Noebels, J., Armstrong, D., Paylor, R. and Zoghbi, H. (2002) “Mice with truncated MeCP2 recapitulate many Rett syndrome features and display hyperacetylation of histone H3.,” Neuron, 35(2), pp. 243-254. doi: 10.1016/s0896-6273(02)00768-7.
Shahbazian, M. D., Antalffy, B., Armstrong, D. L. and Zoghbi, H. Y. (2002) “Insight into Rett syndrome: MeCP2 levels display tissue- and cell-specific differences and correlate with neuronal maturation.,” Human Molecular Genetics, 11(2), pp. 115-124. doi: 10.1093/hmg/11.2.115.
Shen, S., Picci, C., Ustinova, K., Benoy, V., Kutil, Z., Zhang, G., Tavares, M. T., Pavlíček, J., Zimprich, C. A., Robers, M. B., Van Den Bosch, L., Barinka, C., Langley, B. and Kozikowski, A. P. (2021) “Tetrahydroquinoline-Capped Histone Deacetylase 6 Inhibitor SW-101 Ameliorates Pathological Phenotypes in a Charcot-Marie-Tooth Type 2A Mouse Model.,” Journal of Medicinal Chemistry. doi: 10.1021/acs.jmedchem.0c02210.
Shovlin, S. and Tropea, D. (2018) “Transcriptome level analysis in Rett syndrome using human samples from different tissues.,” Orphanet Journal of Rare Diseases, 13(1), p. 113. doi: 10.1186/s13023-018-0857-8.
Singh, A. K., Halder-Sinha, S., Clement, J. P. and Kundu, T. K. (2018) “Epigenetic modulation by small molecule compounds for neurodegenerative disorders.,” Pharmacological Research, 132, pp. 135-148. doi: 10.1016/j.phrs.2018.04.014.
Subramanian, K., Rauniyar, N., Lavalleé-Adam, M., Yates, J. R. and Balch, W. E. (2017) “Quantitative Analysis of the Proteome Response to the Histone Deacetylase Inhibitor (HDACi) Vorinostat in Niemann-Pick Type C1 disease.,” Molecular & Cellular Proteomics, 16(11), pp. 1938-1957. doi: 10.1074/mcp.M116.064949.
Tarquinio, D. C., Hou, W., Neul, J. L., Kaufmann, W. E., Glaze, D. G., Motil, K. J., Skinner, S. A., Lee, H.-S. and Percy, A. K. (2015) “The Changing Face of Survival in Rett Syndrome and MECP2-Related Disorders.,” Pediatric Neurology, 53(5), pp. 402-411. doi: 10.1016/j.pediatrneurol.2015.06.003.
Tsuchiya, Y., Minami, Y., Umemura, Y., Watanabe, H., Ono, D., Nakamura, W., Takahashi, T., Honma, S., Kondoh, G., Matsuishi, T. and Yagita, K. (2015) “Disruption of MeCP2 attenuates circadian rhythm in CRISPR/Cas9-based Rett syndrome model mouse.,” Genes To Cells, 20(12), pp. 992-1005. doi: 10.1111/gtc.12305.
Vashi, N. and Justice, M. J. (2019) “Treating Rett syndrome: from mouse models to human therapies.,” Mammalian Genome, 30(5-6), pp. 90-110. doi: 10.1007/s00335-019-09793-5.
Wang, J., Xiao, Y., Liu, C., Huang, Y., Petersen, R. B., Zheng, L. and Huang, K. (2021) “Emerging physiological and pathological roles of MeCP2 in non-neurological systems.,” Archives of Biochemistry and Biophysics, 700, p. 108768. doi: 10.1016/j.abb.2021.108768.
Watkins, P. A., Maiguel, D., Jia, Z. and Pevsner, J. (2007) “Evidence for 26 distinct acyl-coenzyme A synthetase genes in the human genome.,” Journal of Lipid Research, 48(12), pp. 2736-2750. doi: 10.1194/jlr.M700378-JLR200.
Wither, R. G., Lang, M., Zhang, L. and Eubanks, J. H. (2013) “Regional MeCP2 expression levels in the female MeCP2-deficient mouse brain correlate with specific behavioral impairments.,” Experimental Neurology, 239, pp. 49-59. doi: 10.1016/j.expneurol.2012.09.005.
Wong, L. C., Chen, Y.-T., Tsai, S.-M., Lin, Y.-J., Hsu, C.-J., Wang, H.-P., Hu, S.-C., Shen, H.-Y., Tsai, W.-C. and Lee, W.-T. (2021) “Dietary intake and growth deficits in Rett syndrome-A cross-section study.,” Autism research: official journal of the International Societyfor Autism Research, 14(7), pp. 1512-1521. doi: 10.1002/aur.2508.
Wu, Y., Zhong, W., Cui, N., Johnson, C. M., Xing, H., Zhang, S. and Jiang, C. (2016) “Characterization of Rett Syndrome-like phenotypes in Mecp2-knockout rats.,” Journal of neurodevelopmental disorders, 8, p. 23. doi: 10.1186/s11689-016-9156-7.
Zhang, X., Bao, X., Zhang, Jingjing, Zhao, Y., Cao, G., Pan, H., Zhang, Jie, Wei, L. and Wu, X. (2012) “Molecular characteristics of Chinese patients with Rett syndrome.,” European Journal of Medical Genetics, 55(12), pp. 677-681. doi: 10.1016/j.ejmg.2012.08.009.
Zhang, X., Lin, J.-S. and Spruyt, K. (2021) “Sleep problems in Rett syndrome animal models: A systematic review.,” Journal of Neuroscience Research, 99(2), pp. 529-544. doi: 10.1002/jnr.24730.

1-23. (canceled)

24. A composition comprising a compound according to: 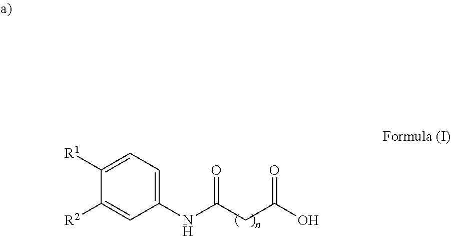
wherein R¹ and R² can be the same or different and
R¹ is H or 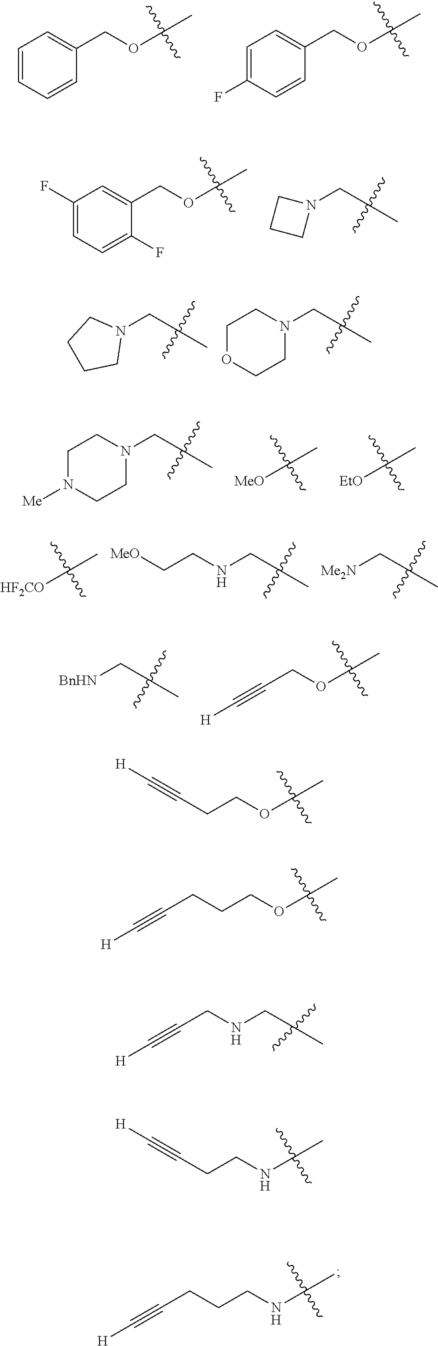
R² is H or 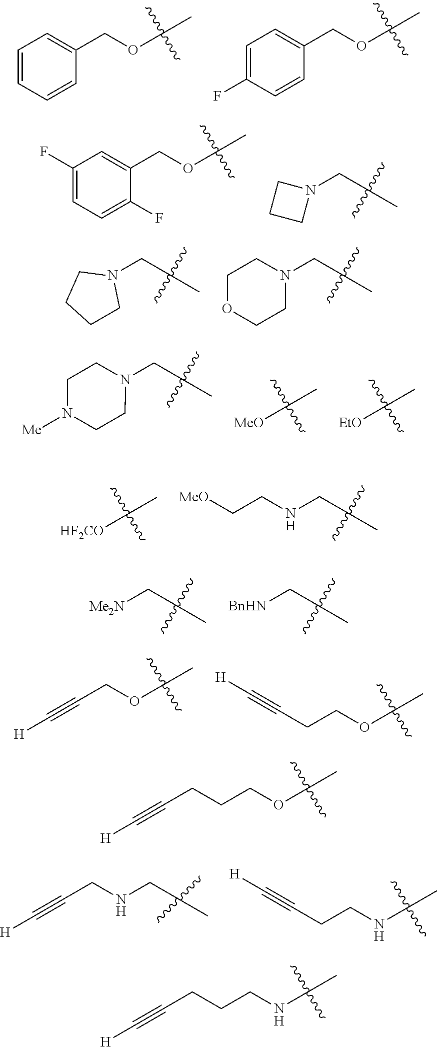
and n is 1 to 12, with the proviso that when R¹ and R² are both H, n is 1-4 or 6-12; 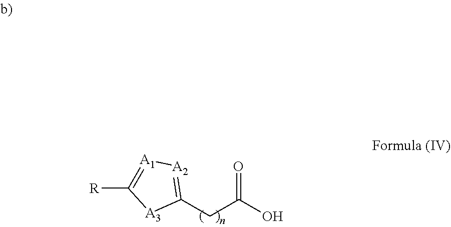
wherein R is H or 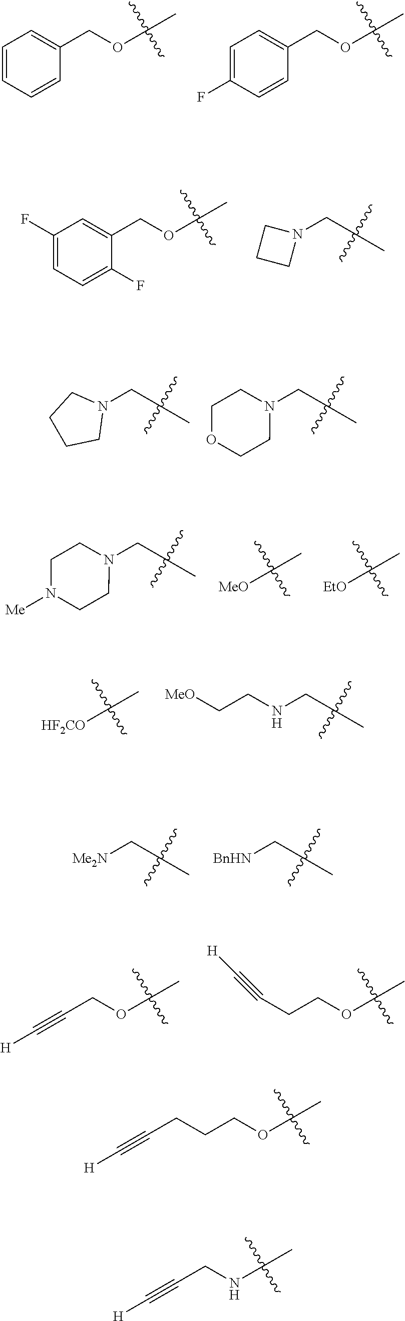 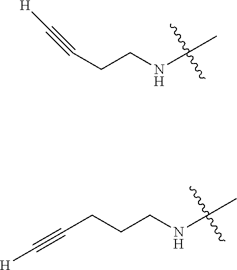 [SMILES: *NCCC#C ; *NCCCC#C]
A₁ is N or CH;
A₂ is N or CH;
A₃ is O or S;
and n is 1 to 12; 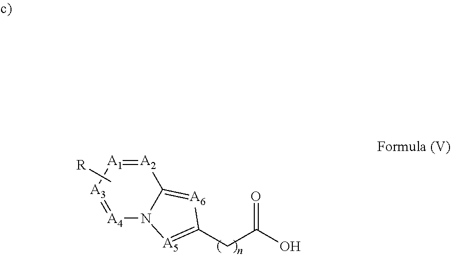
wherein R is H or 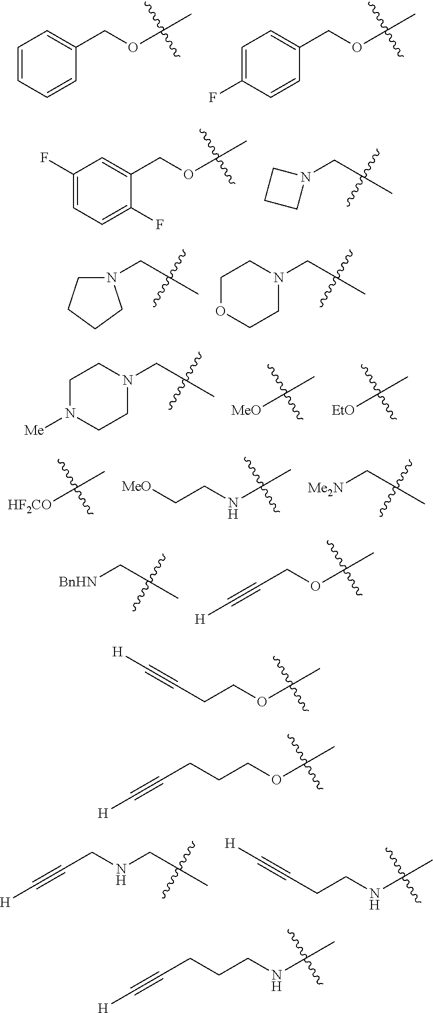
A₁ is N or CH;
A₂ is N or CH;
A₃ is N or CH;
A₄ is N or CH;
A₅ is N or CH;
A₆ is N or CH;
and n is 1 to 12.

25. The composition of claim 24, wherein the compound is according to Formula (VI), Formula (VII), or Formula (VIII): 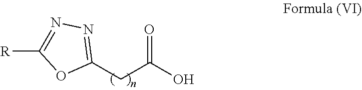
wherein R is H or 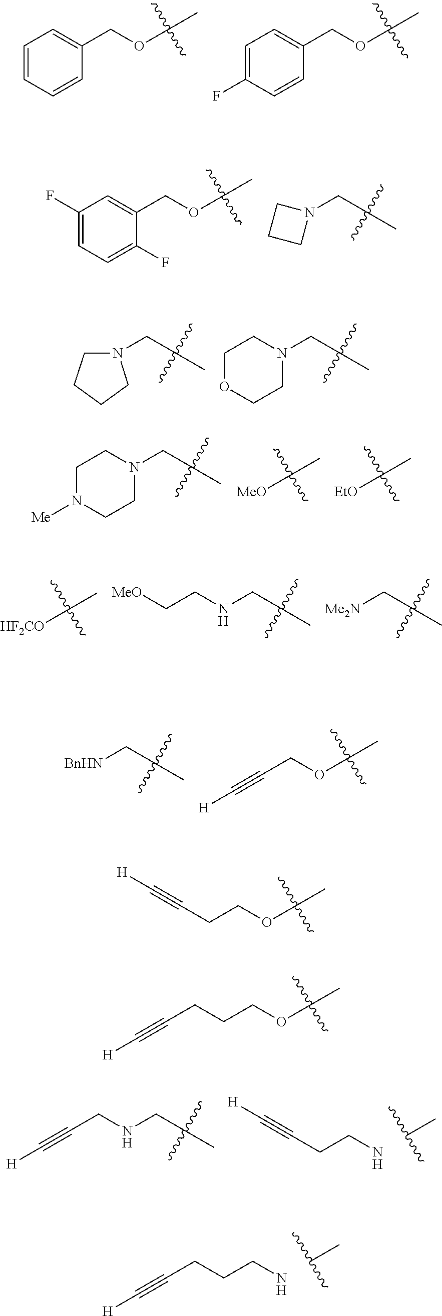
and n is an integer from 1 to 12; 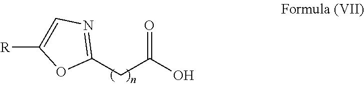
wherein R is H or 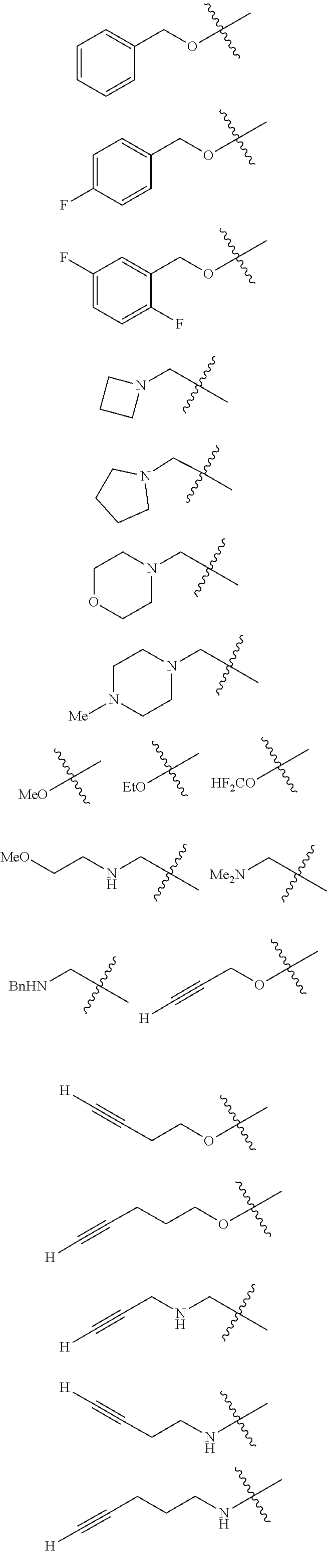
and n is an integer from 1 to 12; 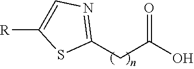
Formula (VIII)
wherein R is H or 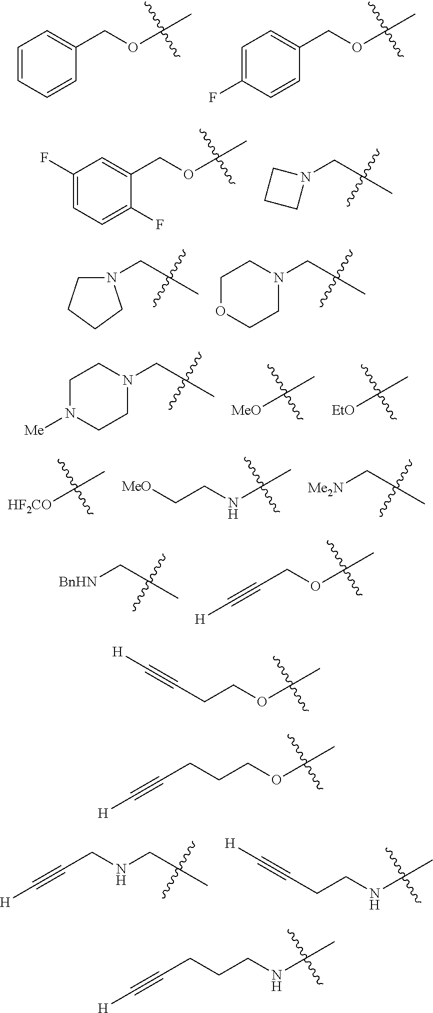
and n is an integer from 1 to 12.

26. The composition of claim 24, wherein the compound is according to Formula (IX), Formula (X), or Formula (XI):
Formula (IX) 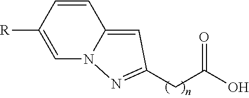
wherein R is H or 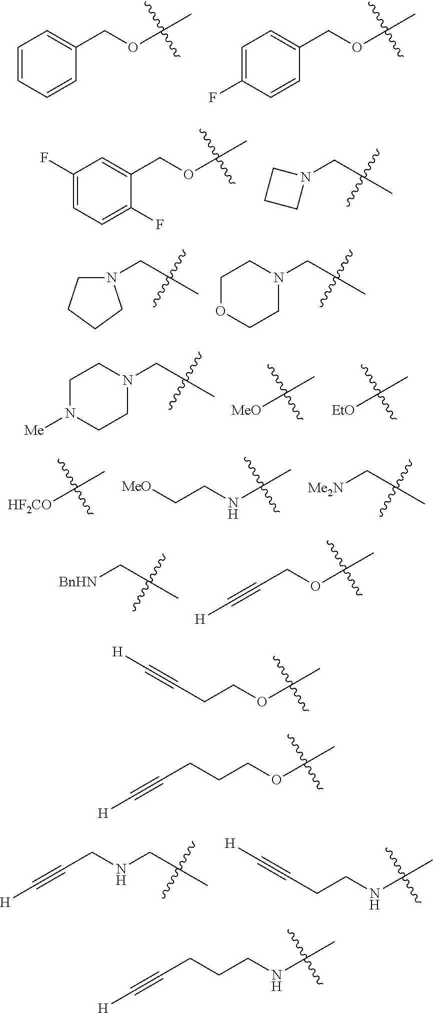
and n is an integer from 1 to 12; 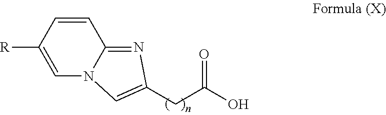
wherein R is H or 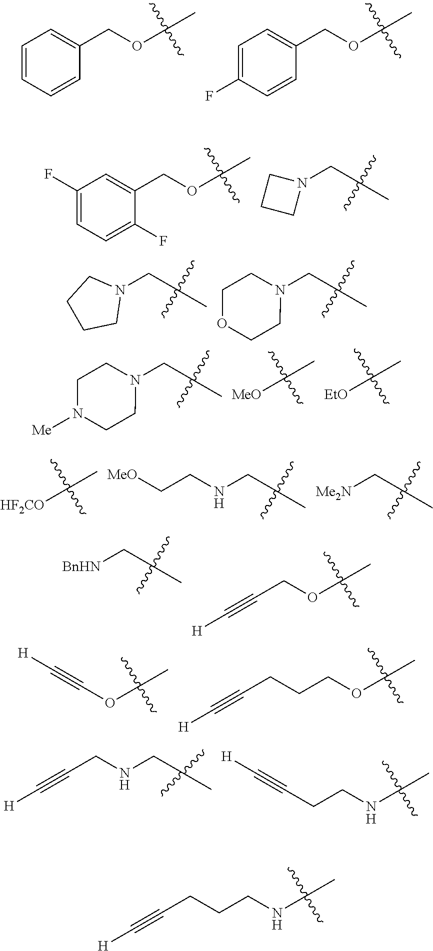
and n is an integer from 1 to 12; 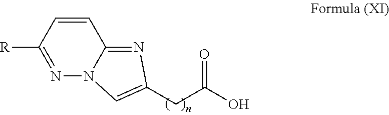
wherein R is H or 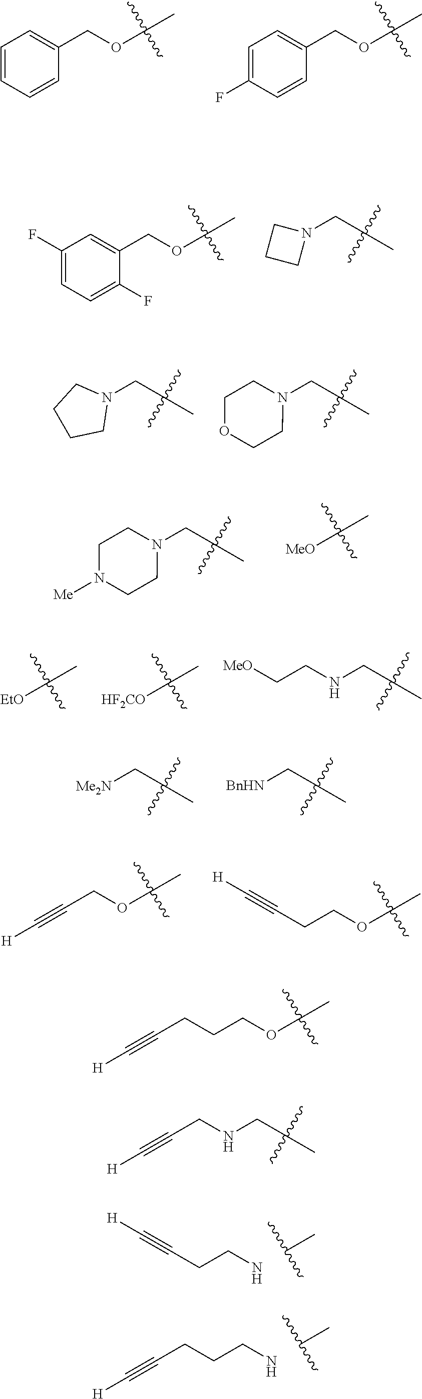
and n is an integer from 1 to 12.

27. A method of treating a subject with a loss-of-function mutation of a gene encoding methyl-CpG binding protein (MeCP2), comprising administering a composition according to claim 24, said composition comprising a compound of Formula (I) to the subject 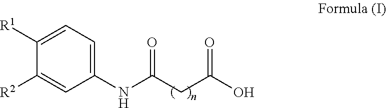
wherein R¹ and R² can be the same or different and
R¹ is H or 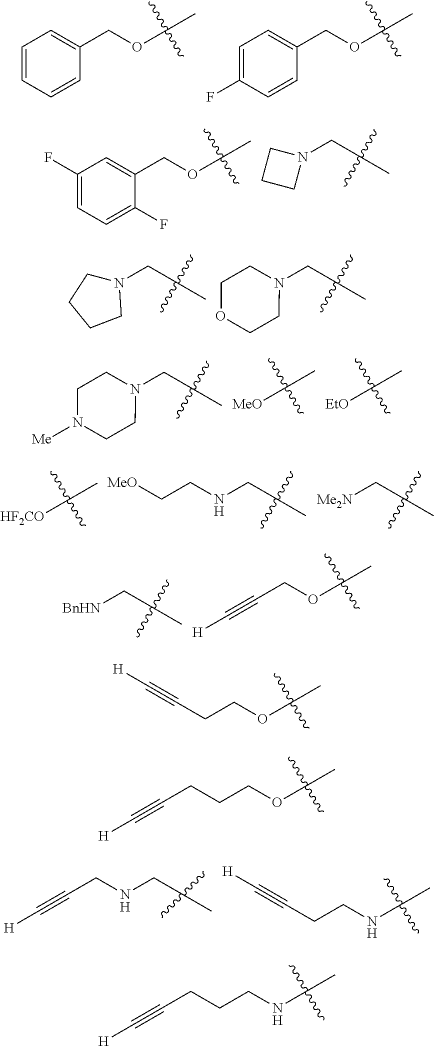
R² is H or 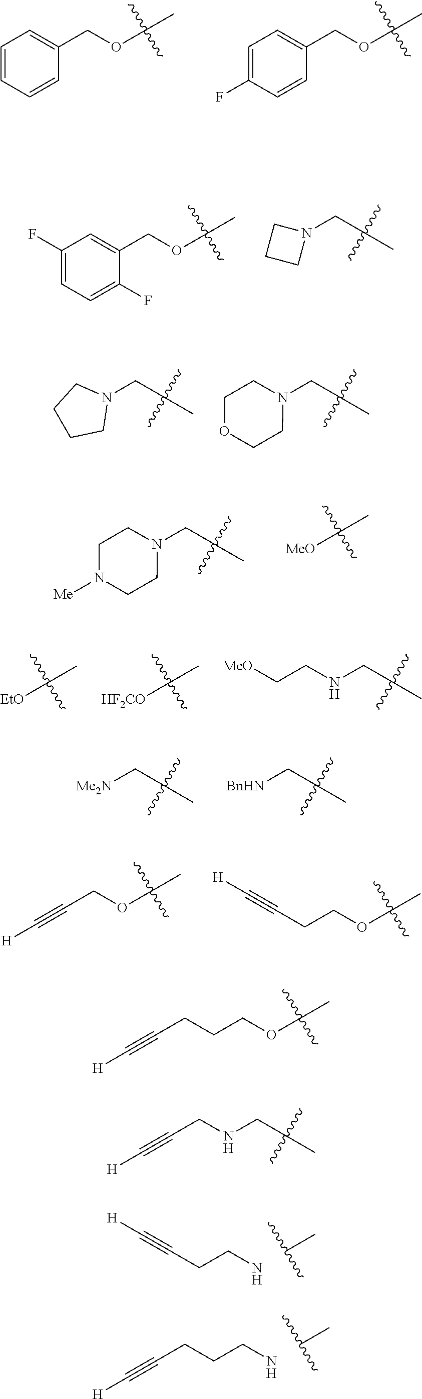
and n is 1 to 12, with the proviso that when R¹ and R² are both H, n is 1-4 or 6-12.

28. The method of claim 27, wherein the subject has Rett syndrome, Fragile X syndrome, or Leigh syndrome.

29. The method of claim 27, wherein the compound is succinanilic acid (RVL002), according to Formula (II)

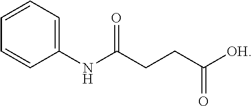 [SMILES: O=C(O)CCC(=O)Nc1ccccc1]

30. A method of treating a subject with a loss-of-function mutation of a gene encoding methyl-CpG binding protein (MeCP2), comprising administering a composition according to claim 24 to the subject, said composition comprising the compound of 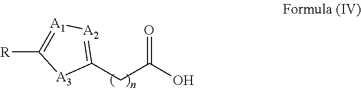
wherein R is H or 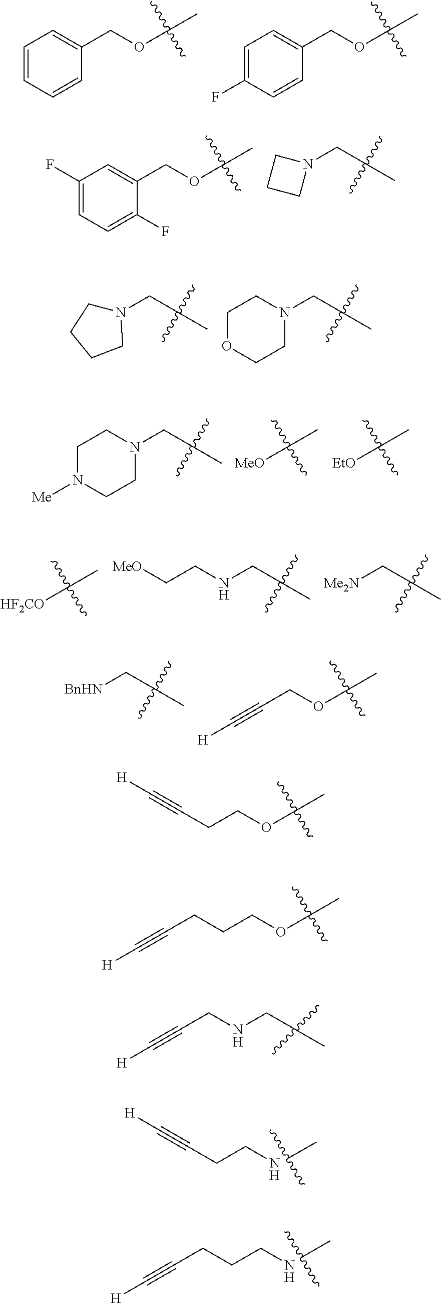
A₁ is N or CH;
A₂ is N or CH;
A₃ is O or S;
and n is 1 to 12.

31. The method of claim 30, wherein the subject has Rett syndrome, Fragile X syndrome, or Leigh syndrome.

32. The method of claim 30, wherein the compound is according to Formula (VI), Formula (VII), or Formula (VIII): 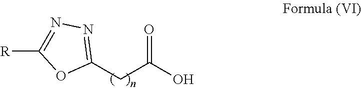
wherein R is H or 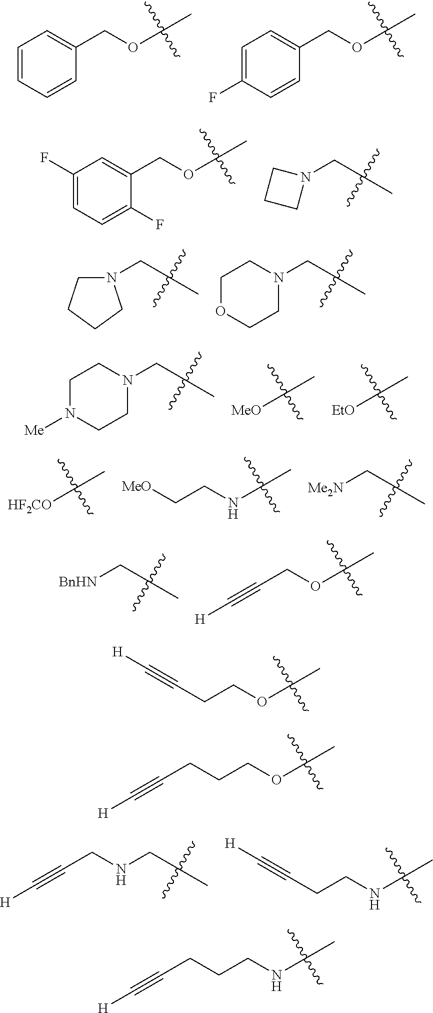
and n is an integer from 1 to 12; 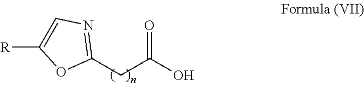
wherein R is H or 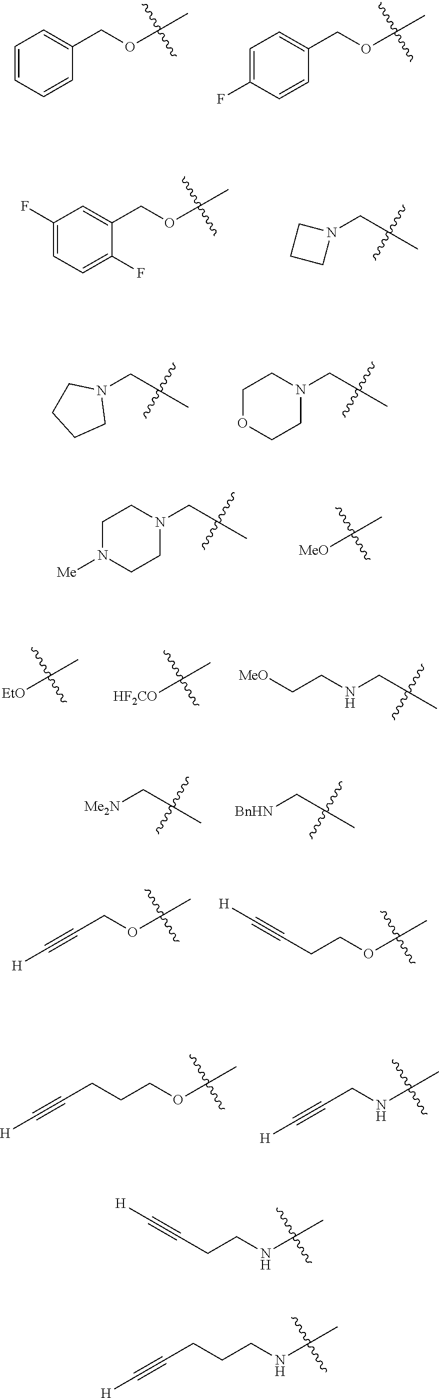
and n is an integer from 1 to 12; 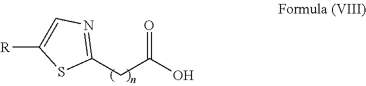
wherein R is H or 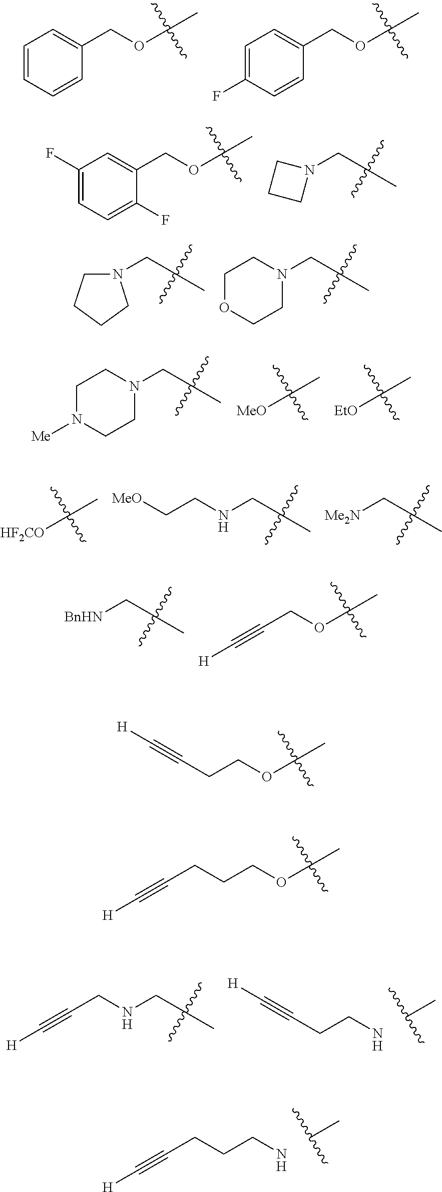
and n is an integer from 1 to 12.

33. A method of treating a subject with a loss-of-function mutation of a gene encoding methyl-CpG binding protein (MeCP2), comprising administering a composition comprising a compound according to claim 25, said composition comprising the compound of Formula (V), to the subject 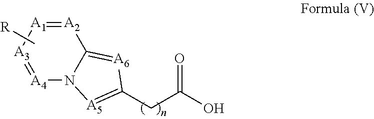
wherein R is H or 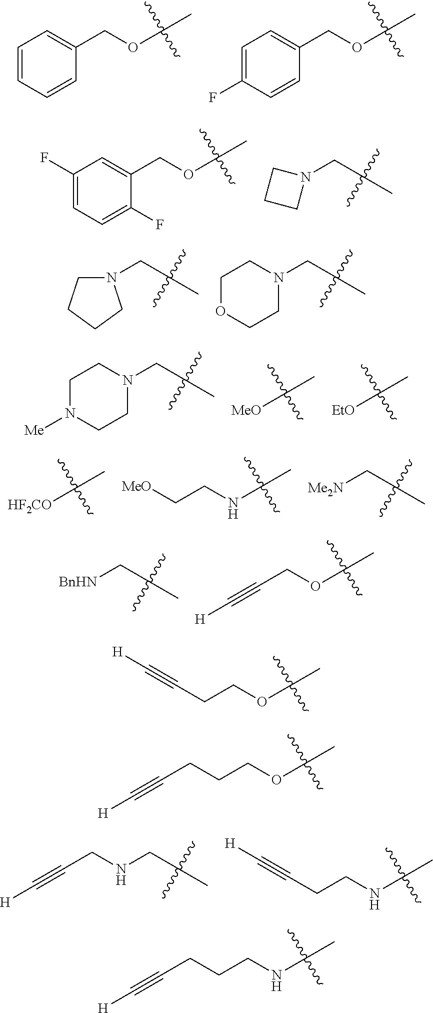
A₁ is N or CH;
A₂ is N or CH;
A₃ is N or CH;
A₄ is N or CH;
A₅ is N or CH;
A₆ is N or CH;
and n is 1 to 12.

34. The method of claim 33, wherein the subject has Rett syndrome, Fragile X syndrome, or Leigh syndrome.

35. The method of claim 33, wherein the compound is according to Formula (IX), Formula (X), or Formula (XI): 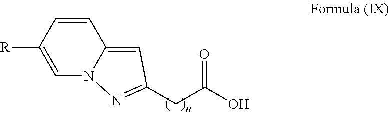
wherein R is H 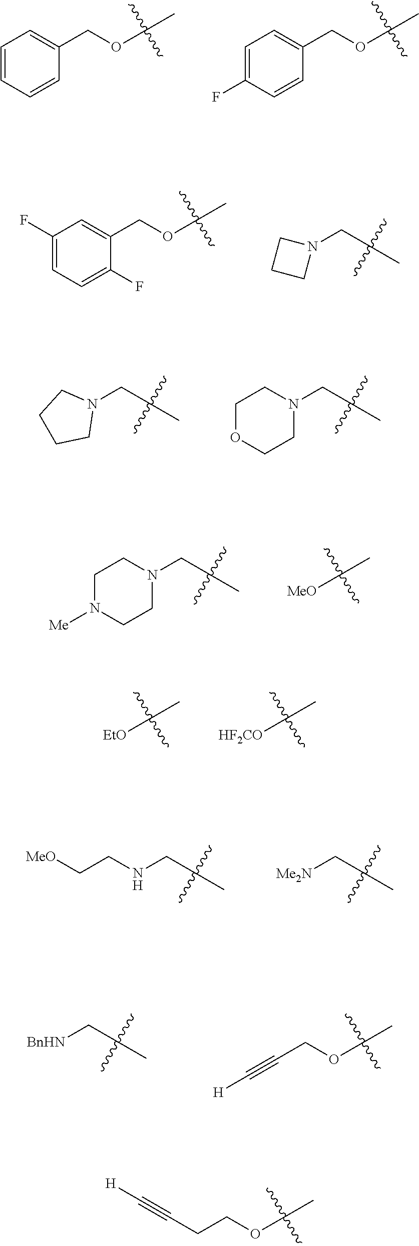 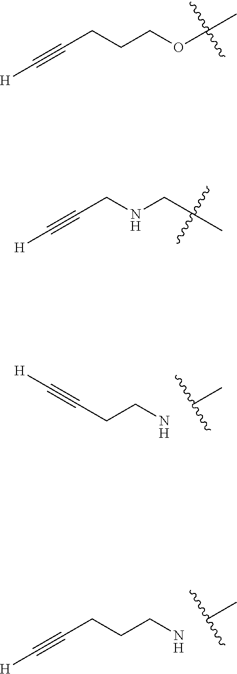
and n is an integer from 1 to 12; 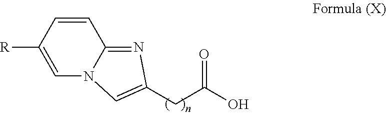
wherein R is H or 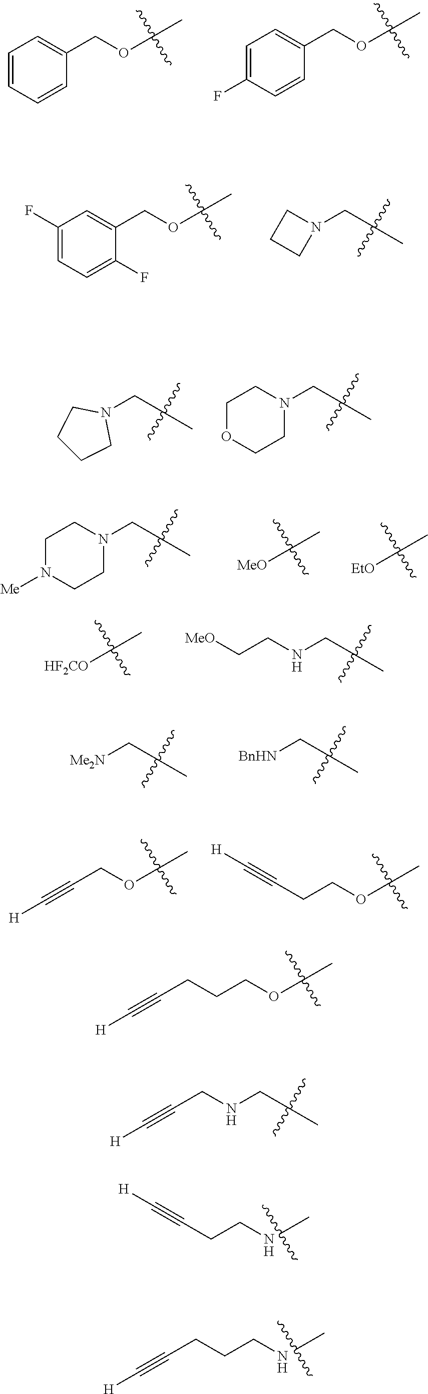
and n is an integer from 1 to 12; 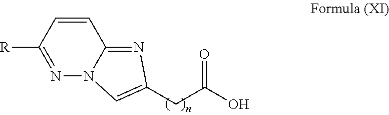
wherein R is H or 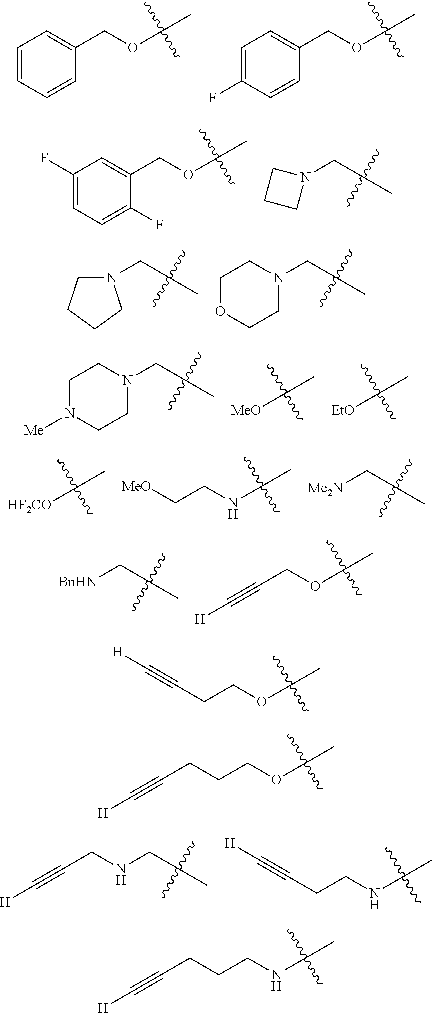
and n is an integer from 1 to 12.

36. A method of treating a disease or disorder comprising the administration of composition according to claim 24 to a subject having a disease or disorder selected from the group consisting of: fatty liver/NAFLD/NASH/MASH steatosis, fibrosis, and inflammation states; obesity; DM1, KMT2B; Parkinson's disease; Pitt Hopkins syndrome; Leigh syndrome, mutations in MT-ND1, SURF1, or other Complex IV genes; MEF2C haploinsufficiency; CIC haploinsufficiency; Fragile X syndrome; AP-4 neurodevelopmental disorder; FOXG1 syndrome; CDKL5 Deficiency Disorder; disorders caused by a deletion or mutation of the SHANK3 gene, Phelan-McDermid syndrome; and other Rett-like disorders.

37. The method of claim 36, wherein said compound or composition is administered in combination with an additional therapeutic agent for the treatment of said disease or disorder.

38. The method of claim 36, wherein the compound is administered in amounts that range from about 0.01 to about 1,000 mg/kg of a subject to be treated or in amounts that can range between about 0.25 and about 5 mg/kg/day, between about 0.4 and 4.5 mg/kg/day, between about 0.6 and 4.25 mg/kg/day, between about 0.8 and 4 mg/kg/day, between about 1 and 3.75 mg/kg/day, between about 0.75 and 3 mg/kg/day, between about 0.5 and 2.75 mg/kg/day, or between about 0.45 and 2.5 mg/kg/day.

39. The method of claim 27, wherein the compound is administered in amounts that range from about 0.01 to about 1,000 mg/kg of a subject to be treated or in amounts that can range between about 0.25 and about 5 mg/kg/day, between about 0.4 and 4.5 mg/kg/day, between about 0.6 and 4.25 mg/kg/day, between about 0.8 and 4 mg/kg/day, between about 1 and 3.75 mg/kg/day, between about 0.75 and 3 mg/kg/day, between about 0.5 and 2.75 mg/kg/day, or between about 0.45 and 2.5 mg/kg/day.

40. The method of claim 38, wherein the compound is succinanilic acid.

41. The method of claim 39, wherein the compound is succinanilic acid.
